(19) United States
(12) Patent Application Publication
(10) Pub. No.: US 2026/0287508 A1
(43) Pub. Date: Sep. 24, 2026

(54) POSITIVE DETERMINATION METHOD, IMAGE ANALYSIS SYSTEM, AND INFORMATION PROCESSING APPARATUS

(71) Applicant: SONY GROUP CORPORATION, TOKYO (JP)

(72) Inventors: SHIORI SASADA, TOKYO (JP); KAZUHIRO NAKAGAWA, TOKYO (JP); KENJI IKEDA, TOKYO (JP)

(21) Appl. No.: 19/168,516

(22) PCT Filed: Mar. 22, 2024
(86) PCT No.: PCT/JP2024/011309

§ 371 Date: Sep. 24, 2025

(30) Foreign Application Priority Data
Apr. 5, 2023 (JP) 2023-061391

(51) Int. Cl. G01N 21/64; G06T 7/00

(52) U.S. Cl. CPC G01N 21/6428; G01N 21/6458; G06T 7/0014; G01N 2021/6439; G01N 2021/646; G06T 2207/10056; G06T 2207/10064; G06T 2207/20081; G06T 2207/30024

(57) ABSTRACT

A positive determination method according to the present disclosure includes a first analysis step of performing first analysis processing including cell detection on a stained image obtained by imaging a stained specimen prepared by staining a specimen containing a target cell with a fluorescent reagent, a second analysis step of performing second analysis processing, which is the same as the first analysis processing, on an unstained image obtained by imaging an unstained specimen, the unstained specimen containing the target cell and being the same as and/or similar to the specimen, the first analysis step and the second analysis step being executed by a processor, and the method includes outputting an index indicating whether or not the target cell is a positive cell, based on a difference between an analysis result of the first analysis processing and an analysis result of the second analysis processing.

### TECHNICAL FIELD

The present disclosure relates to a positive determination method, an image analysis system, and an information processing apparatus.

### BACKGROUND ART

As a method of performing a positive determination, a method based on a multi-stained image obtained by imaging a sample fluorescently stained with a plurality of fluorescent pigments having different emission wavelengths is known (for example, PTL 1 to PTL 3).

### CITATION LIST

### Patent Literature

PTL 1: US 2011/13288665 A
PTL 2: US 2017/15448550 A
PTL 3: US 2016/15396536 A

### SUMMARY

### Technical Problem

In the positive determination of a multi-stained image in the existing technology, the influences of leakage of autofluorescence of a cell and spatial leakage of an adjacent cell contribute to inaccurate determination. Therefore, a threshold for the positive determination depends on visual check at present, and objective and accurate positive determination cannot be achieved.

An object of the present disclosure is to provide a positive determination method, an image analysis system, and an information processing apparatus capable of performing a positive determination, based on an objective index.

### Solution to Problem

A positive determination method according to the present disclosure includes a first analysis step of performing first analysis processing including cell detection on a stained image obtained by imaging a stained specimen prepared by staining a specimen containing a target cell with a fluorescent reagent, and a second analysis step of performing second analysis processing that is the same as the first analysis processing on an unstained image obtained by imaging an unstained specimen, the unstained specimen containing the target cell and being the same as and/or similar to the specimen, the first analysis step and the second analysis step being executed by a processor, and the method includes outputting an index indicating whether or not the target cell is a positive cell, based on a difference between an analysis result of the first analysis processing and an analysis result of the second analysis processing.

### BRIEF DESCRIPTION OF DRAWINGS

FIG. 1 is a block diagram depicting a configuration example of an information processing system applicable to an embodiment of the present disclosure.

FIG. 2 is a schematic diagram depicting specific examples of fluorescence spectra acquired by a fluorescence signal acquisition unit.

FIG. 3 is a schematic diagram depicting specific examples of fluorescence spectra acquired by the fluorescence signal acquisition unit.

FIG. 4 is a diagram depicting fluorescence spectra of AF546 and AF555 when a wavelength resolution is 8 nm.

FIG. 5 is a diagram depicting fluorescence spectra of AF546 and AF555 when the wavelength resolution is 1 nm.

FIG. 6 is a diagram depicting an example of a linked fluorescence spectrum generated from the fluorescence spectra acquired by the fluorescence signal acquisition unit.

FIG. 7 is a block diagram depicting a more specific configuration example of a separation processing unit applicable to each embodiment.

FIG. 8 is a schematic diagram depicting a specific example of a linked autofluorescence reference spectrum when autofluorescent substances are Hemoglobin, Archidonic Acid, Catalase, Collagen, FAD, NADPH, and ProLong Diamond.

FIG. 9 is a schematic diagram for describing measurement channels.

FIG. 10 is a schematic diagram depicting a configuration example of a microscope system when the information processing system applicable to each embodiment is implemented as the microscope system.

FIG. 11 is an example of a flowchart depicting an example of a series of processing flow involved in fluorescence separation by an information processing apparatus.

FIG. 12 is a block diagram depicting an example of a hardware configuration of the information processing apparatus applicable to each embodiment.

FIG. 13 is an example of a flowchart depicting a positive determination method according to a first embodiment.

FIG. 14 is a schematic diagram for describing color separation processing applied to the first embodiment and color separation processing using a virtual filter.

FIG. 15 is a schematic diagram depicting results of performing color separation by using the virtual filter and results of performing by using color separation applied to the first embodiment in comparison with each other.

FIG. 16 is a schematic diagram for describing erroneous determination due to spatial leakage.

FIG. 17 is a schematic diagram for describing an average luminance method according to an existing technology.

FIG. 18 is a schematic diagram for describing a positive pixel method according to the first embodiment.

FIG. 19 is a schematic diagram for describing an on-membrane luminance continuity calculation method according to the first embodiment.

FIG. 20 is a schematic diagram for more specifically describing the on-membrane luminance continuity calculation method according to the first embodiment.

FIG. 21 is an example of a flowchart depicting processing by the on-membrane luminance continuity calculation method according to the first embodiment.

FIG. 22 is a schematic diagram for describing linking of luminance values.

FIG. 23 is a schematic diagram depicting actual data examples of determination results by each positive determination method.

FIG. 24 is a schematic diagram depicting examples of TP, FP, FN, and TN, as well as sensibility, specificity, and accuracy in each of the average luminance method, the positive pixel method, a combination of the average luminance method and the positive pixel method, and the on-membrane luminance continuity calculation method.

FIG. 25 is a schematic diagram for describing an example of a flow of positive determination processing according to a second embodiment.

FIG. 26 is a schematic diagram depicting an example of a detection result when cell detection is performed without performing nucleus detection.

### DESCRIPTION OF EMBODIMENTS

Hereinafter, embodiments of the present disclosure will be described in detail with reference to the drawings. In the following embodiments, the same components are denoted by the same reference signs, and redundant description thereof will be omitted.

Hereinafter, the embodiments of the present disclosure will be described in the following order.
1. Existing Technology
2. Configuration Applicable to Embodiments
3. First Embodiment
3-1. Processing According to First Embodiment
3-2. Threshold Setting Using Unstained Image without Depending on Subjectivity
3-3. Positive Determination Method Capable of Suppressing Spatial Leakage of Cell
3-4. Examples of Determination Results by Each Positive Determination Method
4. Second Embodiment

### 1. EXISTING TECHNOLOGY

The existing technology according to the present disclosure will be described.

As a method of performing a positive determination, a method based on a multi-stained image obtained by imaging a sample fluorescently stained with a plurality of fluorescent pigments having different emission wavelengths is known. In the positive determination in the multi-stained image, the influences of leakage of autofluorescence of a cell and spatial leakage of an adjacent cell contribute to inaccurate determination. Therefore, a threshold for the positive determination is determined depending on visual check at present, and objective and accurate positive determination cannot be achieved.

On the other hand, cells in a tissue do not exist individually in isolation, but are adjacent to each other. Therefore, in order to more accurately determine whether each cell is positive, it is necessary to subjectively determine a likely threshold while viewing a tissue image. In more detail, in the existing technology, a positive determination has been performed by an operation in which a doctor or a researcher manually inputs a threshold for the positive determination and visually checks an optimal threshold for a cell population. This method imposes a time burden on the operator, and the results may vary (reproducibility may not be maintained) due to subjectivity.

Positive cells refer to cells expressing or having an intended molecular target such as a specific biomarker, gene, or protein. These cells can be distinguished from negative cells (cells not expressing or having an intended molecular target) by using a specific detection method or a testing technique such as an immunostaining method using a fluorescence-labeled antibody, a polymerase chain reaction (PCR) method, or western blotting, for example.

The distinguishing between the positive cells and the negative cells is of great significance in research, planning of a therapeutic strategy, and evaluation of a therapeutic effect. The positive cells provide information related to a disease state and a cell function and thus are often used as targets for disease diagnosis and treatment. For example, in cancer cells, the expression states of specific cancer-related genes or proteins are identified as those of positive cells, so that a therapeutic strategy can be selected and a prognosis can be evaluated. In addition, in the diagnosis of an infectious disease, the detection of positive cells indicating the presence of genes or proteins of pathogens can be useful for checking infection and selecting a treatment.

In addition, as described above, cells in a tissue are adjacent to each other, and unlike a system that sorts cells, light leakage from a spatially adjacent cell cannot be avoided in a positive determination method based on a multi-stained image. Thus, in the method according to the existing technology, cases may occur in which a cell that is not originally positive is determined to be positive due to the influence of an adjacent positive cell.

Therefore, in the present disclosure, color separation (autofluorescence removal) and analysis are performed on both of an unstained image and a stained image under the same conditions, so that it is possible to determine an objective threshold related to positive determination. In addition, the present disclosure proposes a luminance information calculation method capable of reducing spatial leakage between adjacent cells, so that it is also possible to determine an objective threshold related to positive determination. Accordingly, the present disclosure makes it possible to accurately and objectively perform a positive determination on a cell in a multi-stained image.

### 2. CONFIGURATION APPLICABLE TO EMBODIMENTS

A configuration applicable to each embodiment of the present disclosure will be described.

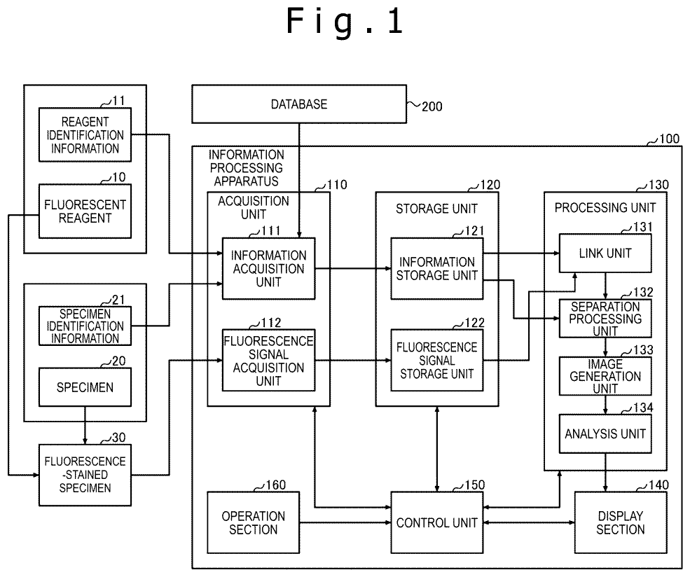

FIG. 1 is a block diagram depicting a configuration example of an information processing system applicable to the embodiment of the present disclosure. As depicted in FIG. 1, the information processing system applicable to the embodiment includes an information processing apparatus 100 and a database 200, and a fluorescent reagent 10, a specimen 20, and a fluorescence-stained specimen 30 are present as inputs to the information processing system.

### Fluorescent Reagent 10

The fluorescent reagent 10 is a chemical agent used for staining the specimen 20. Examples of the fluorescent reagent 10 include a fluorescent antibody (including a primary antibody used for direct labeling or a secondary antibody used for indirect labeling), a fluorescent probe, a nuclear staining reagent, and the like, but the type of fluorescent reagent 10 is not limited thereto. The fluorescent reagent 10 is managed with identification information (hereinafter referred to as “reagent identification information 11”) that allows identification of the fluorescent reagent 10 (or a production lot of the fluorescent reagent 10).

The reagent identification information 11 is, for example, barcode information or the like (one-dimensional barcode information, two-dimensional barcode information, or the like), but is not limited thereto. Even when the same product is used, the property of the fluorescent reagent 10 varies for each production lot depending on the production method, the state of a cell from which an antibody is obtained, or the like. For example, the spectrum, quantum yield, fluorescent labeling ratio, or the like of the fluorescent reagent 10 is different for each production lot. Therefore, in the information processing system, the fluorescent reagent 10 is managed for each production lot by attaching the reagent identification information 11. Thus, the information processing apparatus 100 can perform fluorescence separation in consideration of a slight property difference that appears in each production lot.

### Specimen 20

The specimen 20 is prepared from a biological specimen or a tissue sample collected from a human body for the purpose of pathological diagnosis or the like. The specimen 20 may be a tissue section, a cell, or a fine particle. Regarding the specimen 20, the type of used tissue (for example, organ or the like), the type of target disease, the attribute of a subject (for example, age, sex, blood type, race, or the like), or the lifestyle of the subject (for example, dietary habits, exercise habits, smoking habits, or the like) is not particularly limited. Examples of the tissue section can include a section before staining of a tissue section to be stained (hereinafter, also simply referred to as a section), a section adjacent to a stained section, a section different from a stained section in the same block (sampled from the same place as that of the stained section), a section in a different block in the same tissue (sampled from a different place from that of a stained section), and a section collected from a different patient.

The specimen 20 is managed with identification information (hereinafter referred to as “specimen identification information 21”) that allows identification of the specimen 20. In the same manner as the reagent identification information 11, the specimen identification information 21 is, for example, barcode information or the like (one-dimensional barcode information, two-dimensional barcode information, or the like), but is not limited thereto. The property of the specimen 20 varies depending on the type of used tissue, the type of target disease, the attribute of a subject, the lifestyle of the subject, or the like. For example, the measurement channel, spectrum, or the like of the specimen 20 varies depending on the type of used tissue or the like. In the information processing system, the specimen 20 is individually managed by attaching the specimen identification information 21. This allows the information processing apparatus 100 to perform fluorescence separation in consideration of even a slight property difference that appears in each specimen 20.

### Fluorescence-Stained Specimen 30

The fluorescence-stained specimen 30 is prepared by staining the specimen 20 with the fluorescent reagent 10. In each embodiment, it is assumed that the fluorescence-stained specimen 30 is obtained by staining the specimen 20 with one or more fluorescent reagents 10, but the number of fluorescent reagents 10 used for staining is not particularly limited. A staining method is determined based on a combination of the specimen 20 and the fluorescent reagent 10 but is not particularly limited.

### Information Processing Apparatus 100

As depicted in FIG. 1, the information processing apparatus 100 includes an acquisition unit 110, a storage unit 120, a processing unit 130, a display section 140, a control unit 150, and an operation section 160. The information processing apparatus 100 can be, for example, a fluorescence microscope or the like, but is not necessarily limited thereto and may include various apparatuses. For example, the information processing apparatus 100 may be a personal computer (PC) or the like.

### Acquisition Unit 110

The acquisition unit 110 is configured to acquire information to be used for various types of processing of the information processing apparatus 100. As depicted in FIG. 1, the acquisition unit 110 includes an information acquisition unit 111 and a fluorescence signal acquisition unit 112.

### Information Acquisition Unit 111

The information acquisition unit 111 is configured to acquire information on the fluorescent reagent 10 (hereinafter referred to as “reagent information”) and information on the specimen 20 (hereinafter referred to as “specimen information”). More specifically, the information acquisition unit 111 acquires the reagent identification information 11 attached to the fluorescent reagent 10 used for generating the fluorescence-stained specimen 30 and the specimen identification information 21 attached to the specimen 20. For example, the information acquisition unit 111 acquires the reagent identification information 11 and the specimen identification information 21 using a barcode reader or the like. The information acquisition unit 111 acquires, from the database 200, the reagent information based on the reagent identification information 11 and the specimen information based on the specimen identification information 21. The information acquisition unit 111 stores the acquired information in the information storage unit 121 described below.

Here, in each embodiment, the specimen information includes a linked autofluorescence reference spectrum obtained by linking spectra of autofluorescent substances in the specimen 20 in the wavelength direction, and the reagent information includes a linked fluorescence reference spectrum obtained by linking spectra of fluorescent substances in the fluorescence-stained specimen 30 in the wavelength direction. The linked autofluorescence reference spectrum and the linked fluorescence reference spectrum are collectively referred to as “reference spectra”.

### Fluorescence Signal Acquisition Unit 112

The fluorescence signal acquisition unit 112 is configured to acquire a plurality of fluorescence signals corresponding respectively to a plurality of excitation light beams having different wavelengths when the fluorescence-stained specimen 30 (which is prepared by staining the specimen 20 with the fluorescent reagent 10) is irradiated with the plurality of excitation light beams. More specifically, the fluorescence signal acquisition unit 112 receives light beams and outputs detection signals corresponding to the amounts of the received light beams, thereby acquiring fluorescence spectra of the fluorescence-stained specimen 30, based on the detection signals. Here, the contents of the excitation light (including the excitation wavelength, the intensity, and the like) are determined based on the reagent information and the like (in other words, information on the fluorescent reagent 10 and the like). The fluorescence signal here is not particularly limited as long as it is a signal derived from fluorescence, and may be, for example, a fluorescence spectrum.

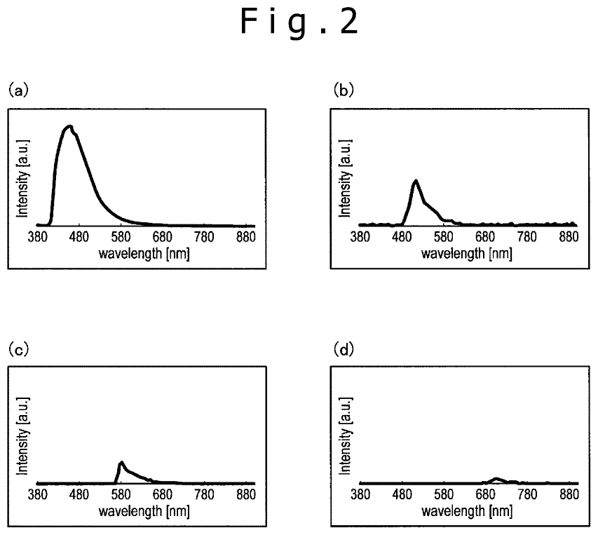

Sections (a) to (d) of FIG. 2 depict specific examples of the fluorescence spectra acquired by the fluorescence signal acquisition unit 112. Sections (a) to (d) of FIG. 2 depict the specific examples of the fluorescence spectra acquired when the fluorescence-stained specimen 30 contains four types of fluorescent substances, namely, DAPI, CK/AF488, PgR/AF594, and ER/AF647 and is irradiated with excitation light beams having, as respective excitation wavelengths, wavelengths of 392 [nm] (Section (a) of FIG. 2), 470 [nm] (Section (b) of FIG. 2), 549 [nm] (Section (c) of FIG. 2), and 628 [nm] (Section (d) of FIG. 2). Note that the fluorescence wavelengths are shifted to a long wavelength side as compared with the excitation wavelengths due to the release of energy for fluorescence emission (Stokes shift). The fluorescent substances contained in the fluorescence-stained specimen 30 and the excitation wavelengths of the emitted excitation light beams are not limited to the above. The fluorescence signal acquisition unit 112 stores the acquired fluorescence spectra in the fluorescence signal storage unit 122 described below.

### Storage Unit 120

The storage unit 120 is configured to store information to be used for various types of processing of the information processing apparatus 100 or information output by various types of processing. As depicted in FIG. 1, the storage unit 120 includes an information storage unit 121 and a fluorescence signal storage unit 122.

### Information Storage Unit 121

The information storage unit 121 is configured to store the reagent information and the specimen information acquired by the information acquisition unit 111.

### Fluorescence Signal Storage Unit 122

The fluorescence signal storage unit 122 is configured to store the fluorescence signals of the fluorescence-stained specimen 30 acquired by the fluorescence signal acquisition unit 112.

### Processing Unit 130

The processing unit 130 is configured to perform various types of processing including fluorescence separation processing. As depicted in FIG. 1, the processing unit 130 includes a link unit 131, a separation processing unit 132, an image generation unit 133, and an analysis unit 134.

### Link Unit 131

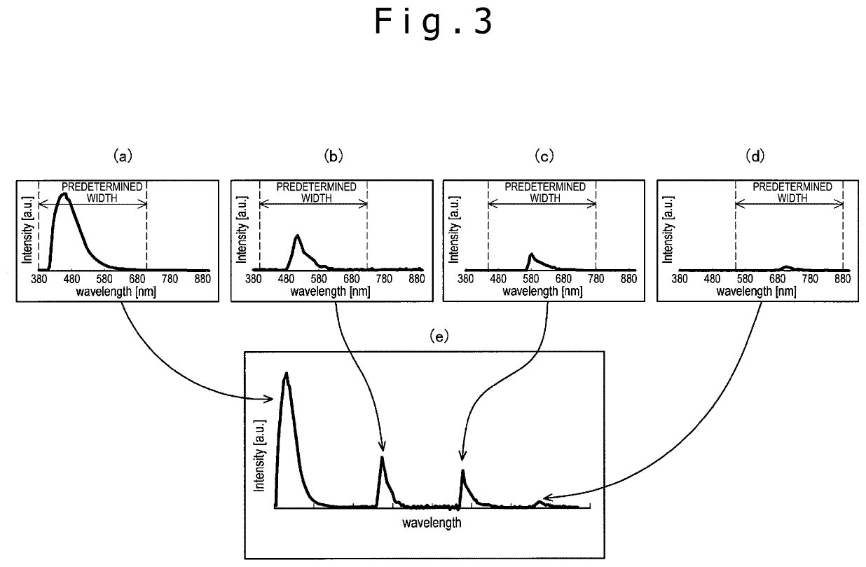

The link unit 131 is configured to generate a linked fluorescence spectrum by linking, in the wavelength direction, at least some of the plurality of fluorescence spectra acquired by the fluorescence signal acquisition unit 112. For example, the link unit 131 extracts data having a predetermined width from each of the four fluorescence spectra (Sections (a) to (d) of FIG. 3) acquired by the fluorescence signal acquisition unit 112 in the above so as to include the maximum value of the fluorescence intensity of each fluorescence spectrum.

The width of the wavelength band from which the link unit 131 extracts the data can be determined based on the reagent information, the excitation wavelength, the fluorescence wavelength, or the like, and may be different for each fluorescent substance. In other words, the width of the wavelength band from which the link unit 131 extracts the data may be different for each of the fluorescence spectra depicted in Sections (a) to (d) of FIG. 3.

Then, as depicted in Section (e) of FIG. 3, the link unit 131 links the extracted pieces of data to each other in the wavelength direction to generate one linked fluorescence spectrum. Note that since the linked fluorescence spectrum includes the pieces of data extracted from the plurality of fluorescence spectra, the wavelength is not continuous at the boundaries of the respective pieces of linked data.

At this time, the link unit 131 performs the above linking after making uniform the intensities of the excitation light beams corresponding respectively to the plurality of fluorescence spectra (in other words, after correcting the plurality of fluorescence spectra), based on the intensities of the excitation light beams. More specifically, the link unit 131 divides the respective fluorescence spectra by the excitation power densities, which are the intensities of the excitation light beams, to perform the above-described linking after making uniform the intensities of the excitation light beams corresponding respectively to the plurality of fluorescence spectra. Thus, the fluorescence spectra when the excitation light beams having the same intensity are emitted are obtained. In addition, when the emitted excitation light beams have different intensities, spectra absorbed by the fluorescence-stained specimen 30 (hereinafter, referred to as “absorption spectra”) also have different intensities depending on the intensities of the excitation light beams. Therefore, as described above, by making uniform the intensities of the excitation light beams corresponding respectively to the plurality of fluorescence spectra, the absorption spectra can be appropriately evaluated.

The intensity of the excitation light in the present description may be the excitation power or the excitation power density as described above. The excitation power or the excitation power density may be power or power density obtained by actually measuring the excitation light emitted from the light source 104, or may be power or power density calculated from a drive voltage applied to the light source 104. The intensity of each excitation light beam in the present description may be a value obtained by correcting the above-described excitation power density with an absorption rate of a section to be observed with respect to each excitation light beam, an amplification factor of a detection signal in a detection system (fluorescence signal acquisition unit 112 or the like) that detects fluorescence emitted from the section, or the like. That is, the intensity of the excitation light beam in the present description may be the power density of the excitation light beam that actually contributes to the excitation of the fluorescent substance, a value obtained by correcting the power density with the amplification factor of the detection system, or the like. By taking into account the absorption rate, the amplification factor, or the like, it is possible to appropriately correct the intensity of the excitation light that changes with change in the machine state, the environment, or the like. Thus, it is possible to generate a linked fluorescence spectrum that enables color separation with higher accuracy.

The correction value (also referred to as an intensity correction value) for each fluorescence spectrum based on the intensity of the excitation light is not limited to a value for making uniform the intensity of the excitation light corresponding respectively to the plurality of fluorescence spectra, and may be variously modified. For example, the signal intensity of a fluorescence spectrum having an intensity peak on a long wavelength side tends to be lower than the signal intensity of a fluorescence spectrum having an intensity peak on a short wavelength side. Therefore, when the linked fluorescence spectrum includes both of the fluorescence spectrum having the intensity peak on the long wavelength side and the fluorescence spectrum having the intensity peak on the short wavelength side, the fluorescence spectrum having the intensity peak on the long wavelength side may be hardly taken into account, and only the fluorescence spectrum having the intensity peak on the short wavelength side may be extracted. In such a case, for example, by increasing the intensity correction value for the fluorescence spectrum having the intensity peak on the long wavelength side, it is also possible to increase the separation accuracy of the fluorescence spectrum having the intensity peak on the short wavelength side.

The link unit 131 may correct the wavelength resolutions of the plurality of fluorescence spectra to be linked independently of each other. For example, the fluorescence spectrum of AF546 and the fluorescence spectrum of AF555 have almost the same spectral shape and peak wavelength, and the difference is that the fluorescence spectrum of AF555 has a shoulder at a tail portion on a high wavelength side, whereas the fluorescence spectrum of AF546 does not have such a shoulder. In this way, when two fluorescence spectra are similar, there is a problem in that it is difficult to perform color separation of the two fluorescence spectra by spectrum extraction.

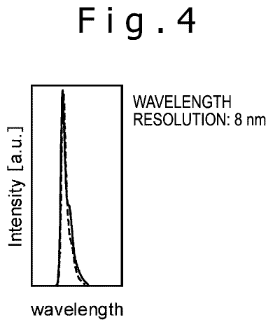

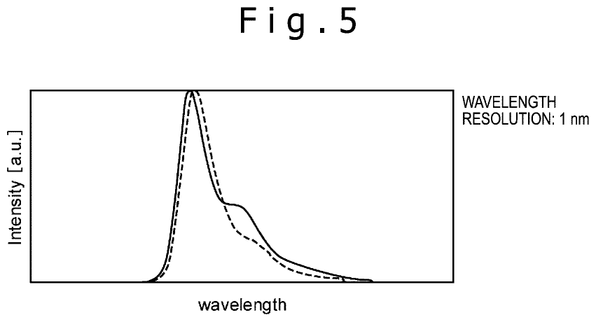

Such a problem may be solved by increasing the wavelength resolution of the linked fluorescence spectrum. FIG. 4 is a diagram depicting the fluorescence spectra of AF546 and AF555 when the wavelength resolution is 8 nm, and FIG. 5 is a diagram depicting the fluorescence spectra of AF546 and AF555 when the wavelength resolution is 1 nm.

As depicted in FIG. 4, when the wavelength resolution is 8 nm, the spectral shape and peak wavelength of AF546 substantially coincide with the spectral shape and peak wavelength of AF555. Therefore, it is practically difficult to perform color separation thereon using, for example, the least squares method.

In contrast, as depicted in FIG. 5, when the wavelength resolution is eight times the wavelength resolution depicted in FIG. 4, that is, when the wavelength resolution is 1 nm, the spectral shape and the peak wavelength of AF546 and the spectral shape and the peak wavelength of AF555 can be clearly separated. This indicates that even when a plurality of fluorescence spectra having similar spectral shapes and peak wavelengths are used, color separation can be performed using these fluorescence spectra by increasing the wavelength resolution.

However, as the wavelength resolution increases, the amount of data of the linked fluorescence spectrum increases, and the required memory capacity, the calculation cost in fluorescence separation processing, and the like increase. Therefore, among the plurality of fluorescence spectra to be linked, the link unit 131 makes a correction so as to increase the wavelength resolution for a fluorescence spectrum assumed to be difficult to be color-separated, and makes a correction so as to decrease the wavelength resolution for a fluorescence spectrum assumed to be easily color-separated. This makes it possible to improve the color separation accuracy while suppressing an increase in the amount of data.

Here, a method of generating the linked fluorescence spectrum by the link unit 131 will be described with reference to a specific example. As in the method of generating the linked fluorescence spectrum described above with reference to FIG. 3, a case of linking four fluorescence spectra obtained by irradiating the fluorescence-stained specimen 30 containing four types of fluorescent substances, namely, DAPI, CK/AF488, PgR/AF594, and ER/AF647, with excitation light beams having, as respective excitation wavelengths, wavelengths of 392 nm, 470 nm, 549 nm, and 628 nm will be described as an example in the present description.

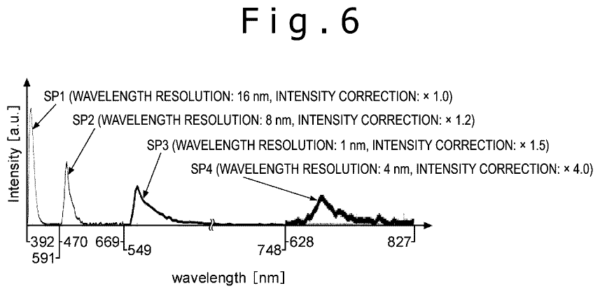

FIG. 6 is a diagram depicting an example of the linked fluorescence spectrum generated from the fluorescence spectra depicted in Sections (a) to (d) of FIG. 3.

As depicted in FIG. 6, the link unit 131 extracts, from the fluorescence spectrum depicted in Section (a) of FIG. 3, a fluorescence spectrum SP1 in a wavelength band in which an excitation wavelength is 392 nm or more and 591 nm or less, extracts, from the fluorescence spectrum depicted in Section (b) of FIG. 3, a fluorescence spectrum SP2 in a wavelength band in which an excitation wavelength is 470 nm or more and 669 nm or less, extracts, from the fluorescence spectrum depicted in Section (c) of FIG. 3, a fluorescence spectrum SP3 in a wavelength band in which an excitation wavelength is 549 nm or more and 748 nm or less, and extracts, from the fluorescence spectrum depicted in Section (d) of FIG. 3, a fluorescence spectrum SP4 in a wavelength band in which an excitation wavelength is 628 nm or more and 827 nm or less.

Next, the link unit 131 corrects the wavelength resolution for the extracted fluorescence spectrum SP1 to 16 nm (without intensity correction), makes a correction to multiply the intensity of the fluorescence spectrum SP2 by 1.2, corrects the wavelength resolution for the fluorescence spectrum SP2 to 8 nm, makes a correction to multiply the intensity of the fluorescence spectrum SP3 by 1.5 (without wavelength resolution correction), makes a correction to multiply the intensity of the fluorescence spectrum SP4 by 4.0, and corrects the wavelength resolution of the fluorescence spectrum SP4 to 4 nm. Then, the link unit 131 sequentially links the corrected fluorescence spectra SP1 to SP4 to generate a linked fluorescence spectrum as depicted in FIG. 6.

FIG. 6 depicts a case where the link unit 131 extracts the fluorescence spectra SP1 to SP4 having a predetermined bandwidth (width of 200 nm in FIG. 6) from the excitation wavelengths when the fluorescence spectra are acquired, and links the fluorescence spectra SP1 to SP4. However, the bandwidths of the fluorescence spectra extracted by the link unit 131 do not need to be the same and may be different. That is, a region extracted from each fluorescence spectrum by the link unit 131 is only required to be a region including the peak wavelength of each fluorescence spectrum, and the wavelength band and the bandwidth thereof may be changed as appropriate. In this case, a shift in the spectral wavelength due to the Stokes shift may be taken into consideration. In this way, by narrowing down the extracted wavelength band, the amount of data can be reduced, and thus the fluorescence separation processing can be performed at a higher speed.

### Separation Processing Unit 132

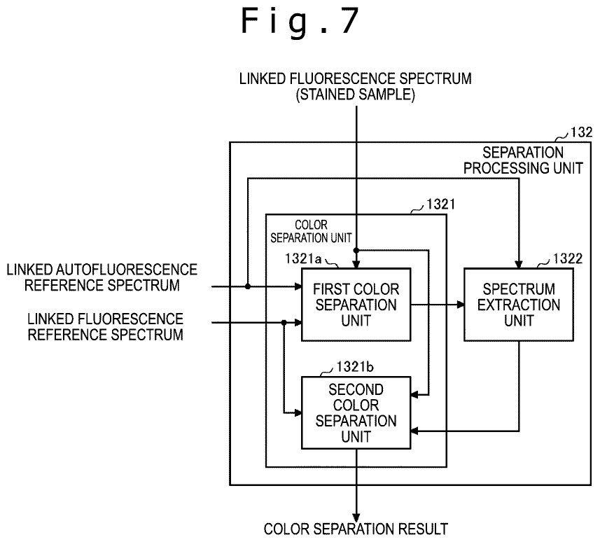

The separation processing unit 132 is configured to separate the linked fluorescence spectrum for each molecule. FIG. 7 is a block diagram depicting a more specific configuration example of the separation processing unit applicable to each embodiment. As depicted in FIG. 7, the separation processing unit 132 includes a color separation unit 1321 and a spectrum extraction unit 1322.

The color separation unit 1321 includes, for example, a first color separation unit 1321a and a second color separation unit 1321b, and performs, for each molecule, color separation on the linked fluorescence spectrum of the stained section (also referred to as a stained sample) input from the link unit 131.

The spectrum extraction unit 1322 is configured to improve the linked autofluorescence reference spectrum so that a color separation result with higher accuracy can be obtained, and adjusts, based on the color separation result of the color separation unit 1321, the linked autofluorescence reference spectrum included in the specimen information input from the information storage unit 121 to a linked autofluorescence reference spectrum with which a color separation result with higher accuracy can be obtained.

More specifically, the first color separation unit 1321a performs color separation processing on the linked fluorescence spectrum of the stained sample input from the link unit 131, using the linked fluorescence reference spectrum included in the reagent information and the linked autofluorescence reference spectrum included in the specimen information, which are input from the information storage unit 121, thereby separating the linked fluorescence spectrum into spectra of respective molecules. Note that, for example, the least squares method (LSM), the weighted least squares method (WLSM), or the like may be used for the color separation processing.

The spectrum extraction unit 1322 performs spectrum extraction processing on the linked autofluorescence reference spectrum input from the information storage unit 121, using the color separation result input from the first color separation unit 1321a, and adjusts the linked autofluorescence reference spectrum based on the result, thereby improving the linked autofluorescence reference spectrum to a linked autofluorescence reference spectrum with which a color separation result with higher accuracy can be obtained. Note that, for example, the non-negative matrix factorization (NMF), the singular value decomposition (SVD), or the like may be used for the spectrum extraction processing.

The second color separation unit 1321b performs color separation processing on the linked fluorescence spectrum of the stained sample input from the link unit 131, using the adjusted linked autofluorescence reference spectrum input from the spectrum extraction unit 1322, thereby separating the linked fluorescence spectrum into spectra of respective molecules. Note that, for example, the least squares method (LSM), the weighted least squares method (WLSM), or the like may be used for the color separation processing in the same manner as the first color separation unit 1321a.

Note that, although FIG. 7 depicts a case where the linked autofluorescence reference spectrum is adjusted once, the present disclosure is not limited thereto, and a final color separation result may be acquired after processing of inputting the color separation result of the second color separation unit 1321b to the spectrum extraction unit 1322 and adjusting the linked autofluorescence reference spectrum again in the spectrum extraction unit 1322 is repeated once or more.

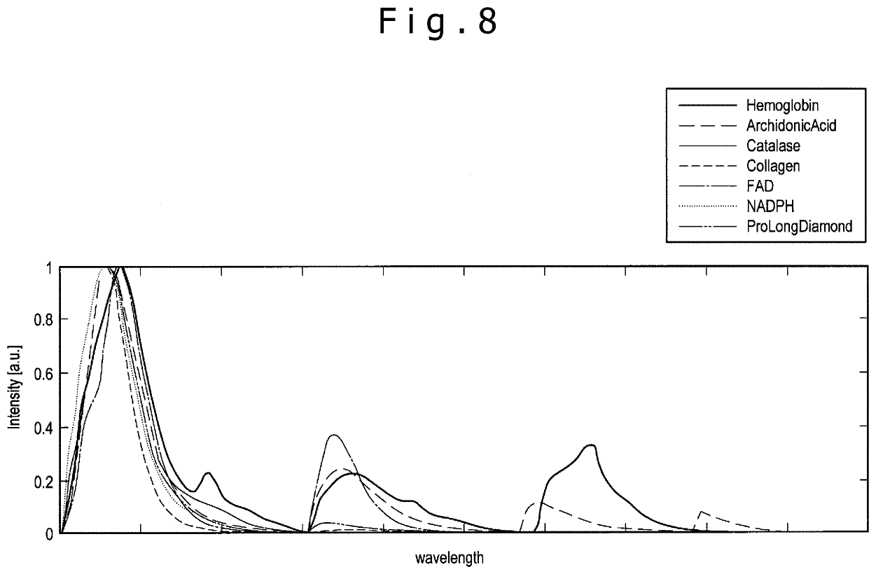

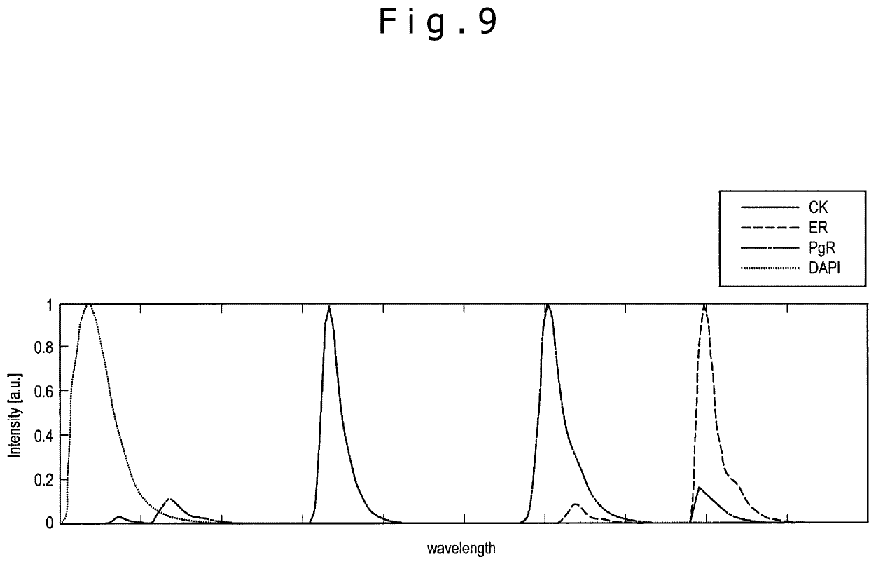

FIG. 8 depicts a specific example of the linked autofluorescence reference spectrum when the autofluorescent substances are Hemoglobin, Archidonic Acid, Catalase, Collagen, FAD, NADPH, and ProLong Diamond. FIG. 9 depicts a specific example of the linked fluorescence reference spectrum when the fluorescent substances are CK, ER, PgR, and DAPI. Both of the linked fluorescence reference spectrum and the linked autofluorescence reference spectrum can be generated by the same method as that of the linked fluorescence spectrum by the link unit 131 (not necessarily limited thereto).

More specifically, the linked fluorescence reference spectrum and the linked autofluorescence reference spectrum can be generated by linking, in the wavelength direction, pieces of data having a predetermined wavelength bandwidth of a plurality of spectra acquired by a plurality of excitation light beams having the same excitation wavelengths as the wavelengths when the linked fluorescence spectrum is generated. At this time, it is assumed that the intensities of the excitation light beams corresponding respectively to the plurality of spectra are made uniform based on the intensities of the excitation light beams (for example, excitation power densities) (not necessarily limited thereto).

The method of generating the linked fluorescence reference spectrum and the linked autofluorescence reference spectrum is not necessarily limited to the above. For example, the linked fluorescence reference spectrum and the linked autofluorescence reference spectrum may be generated based on theoretical values, catalog values, or the like of spectra of respective substances.

Next, calculation related to the least squares method will be described. The least squares method is a method of calculating a color mixture ratio by fitting the linked fluorescence spectrum generated by the link unit 131 to the reference spectra. The color mixture ratio is an index indicating the degree of mixing of each substance. Expression (1) below is an equation representing a residual obtained by subtracting, from the linked fluorescence spectrum (Signal), a spectrum obtained by performing color mixing of the reference spectra (St) (the linked fluorescence reference spectrum and the linked autofluorescence reference spectrum) at a color mixture ratio a. Note that “Signal (1×the number of channels)” in Expression (1) indicates that there are as many linked fluorescence spectra (Signal) as there are wavelength channels (for example, Signal is a matrix representing the linked fluorescence spectrum). Further, “St (the number of substances×the number of channels)” indicates that there are as many reference spectra as there are wavelength channels for each substance (fluorescent substance and autofluorescent substance) (for example, St is a matrix representing the reference spectra). Further, “a (1×the number of substances)” indicates that a color mixture ratio a is set for each substance (fluorescent substance and autofluorescent substance) (for example, a is a matrix representing the color mixture ratio of each reference spectrum in the linked fluorescence spectrum).

$$\begin{matrix}[ Expression 1 & \\ Signal ( 1 \times the number of channels ) - a ( 1 \times the number of substances ) * St ( the number of substances \times the number of channels ) & ( 1 )\end{matrix}$$

Then, the first color separation unit 1321a/the second color separation unit 1321b calculates the color mixture ratio a of each substance at which the sum of squares of Residual Expression (1) is minimized. The sum of squares of the residual is minimized when the result of partial differentiation with respect to the color mixture ratio a in Expression (1) representing the residual is zero. Thus, the first color separation unit 1321a/the second color separation unit 1321b calculates the color mixture ratio a of each substance at which the sum of squares of the residual is minimized by solving following Equation (2). Note that “St′” in Equation (2) indicates a transposed matrix of the reference spectra St. Further, “inv (St*St′)” indicates an inverse matrix of St*St′.

$$\begin{matrix}[ Equation 2 ] & \\ \frac{δ ( Signal - a * St )}{δ a} = 0 \\ ⇔ 2 ( Signal - a * St ) * {St}^{′} = 0 \\ ⇔ ( Signal - a * St ) {St}^{′} = 0 \\ ⇔ Signal * {St}^{′} - a ( St * {St}^{′} ) = 0 \\ a = Signal * {St}^{′} * inv ( St * {St}^{′} ) & ( 2 )\end{matrix}$$

Here, specific examples of values of Expression (1) described above are shown in Equations (3) to (5) below. In the examples of Equations (3) to (5), a case is shown where the reference spectra (St) of three types of substances (the number of substances is three) are mixed at different color mixture ratios a in the linked fluorescence spectrum (Signal).

$$\begin{matrix}[ Equation 3 ] & \\ St = ( \begin{matrix}50 & 100 & 60 & 25 & 4 \\ 10 & 20 & 100 & 20 & 8 \\ 0.1 & 11 & 30 & 100 & 50\end{matrix} ) & ( 3 )\end{matrix}$$

$$\begin{matrix}[ Equation 4 ] & \\ a = ( \begin{matrix}3 & 2 & 1\end{matrix} ) & ( 4 )\end{matrix}$$

$$\begin{matrix}[ Equation 5 ] & \\ Signal = a * St = ( \begin{matrix}170.1 & 351 & 410 & 215 & 78\end{matrix} ) & ( 5 )\end{matrix}$$

A specific example of the calculation result of above-described Equation (2) using the values of Equations (3) and (5) is shown in following Equation (6). As shown in Equation (6), it can be seen that “a=(3 2 1)” (that is, the same value as that of Equation (4)) is correctly calculated as the calculation result.

$$\begin{matrix}[ Equation 6 ] & \\ a = Signal * {St}^{′} * inv ( St * {St}^{′} ) = ( \begin{matrix}3 & 2 & 1\end{matrix} ) & ( 6 )\end{matrix}$$

As described above, the first color separation unit 1321a/the second color separation unit 1321b can output a unique spectrum as a separation result by performing the fluorescence separation processing using the reference spectra (the linked autofluorescence reference spectrum and the linked fluorescence reference spectrum) linked in the wavelength direction (the separation result is not divided into a result for each excitation wavelength). Therefore, the operator can more easily obtain the correct spectrum. In addition, the reference spectrum (linked autofluorescence reference spectrum) related to autofluorescence to be used for separation is automatically acquired, and the fluorescence separation processing is performed, so that the operator does not need to extract a spectrum corresponding to autofluorescence from an appropriate space of the unstained section.

As described above, the first color separation unit 1321a/the second color separation unit 1321b may extract the spectrum of each fluorescent substance from the linked fluorescence spectrum by performing calculation related to the weighted least squares method instead of the least squares method. In the weighted least squares method, by using the fact that noise of the linked fluorescence spectrum (Signal), which is a measurement value, follows the Poisson distribution, weights are added so that an error at a low signal level is emphasized. However, the upper limit value at which the weighting is not performed in the weighted least squares method is set as an Offset value. The Offset value is determined based on the characteristics of a sensor used for measurement, and needs to be optimized separately when an imaging element is used as the sensor. When the weighted least squares method is performed, the reference spectra St in above-described Expression (1) and Equation (2) are replaced with St_ represented by following Equation (7). Note that following Equation (7) means that St_ is calculated by dividing (in other words, element-wise division) each element (each component) of St represented by a matrix by a corresponding element (corresponding component) in “Signal+Offset value” also represented by a matrix.

$$\begin{matrix}[ Equation 7 ] & \\ St = \frac{St}{Signal + Offset Value} & ( 7 )\end{matrix}$$

Here, following Equation (8) shows a specific example of St_ represented by above-described Equation (7) when the Offset value is 1 and the values of the reference spectra St and the linked fluorescence spectrum Signal are represented by above Equations (3) and Formula (5), respectively.

$$\begin{matrix}[ Equation 8 ] & \\ St = \frac{St}{Signal + Offset Value} = ( \begin{matrix}0.2922 & 0.2841 & 0.146 & 0.1157 & 0.0506 \\ 0.0584 & 0.0568 & 0.2433 & 0.0926 & 0.1013 \\ 5.8445 {e}^{- 5} & 0.0313 & 0.073 & 0.463 & 0.6329\end{matrix} ) & ( 8 )\end{matrix}$$

A specific example of the calculation result of the color mixture ratio a in this case is shown in following Equation (9). As shown in Equation (9), it is understood that “a=(3 2 1)” is correctly calculated as the calculation result.

$$\begin{matrix}[ Equation 9 ] & \\ a = Signal * {St}_{-}^{′} * inv ( St * {St}_{-}^{′} ) = ( \begin{matrix}3 & 2 & 1\end{matrix} ) & ( 9 )\end{matrix}$$

The separation processing unit 132 that has separated the fluorescence spectrum and the autofluorescence spectrum performs various types of processing using these spectra.

For example, the separation processing unit 132 may extract a fluorescence spectrum from image information of another specimen 20 by performing subtraction processing (also referred to as “background subtraction processing”) on the image information of the other specimen 20 using the autofluorescence spectrum after the separation.

When there are a plurality of specimens 20 that are the same or similar in terms of a tissue used for the specimens 20, the type of target disease, the attribute of a subject, the lifestyle of the subject, and the like, there is a high possibility that the autofluorescence spectra of these specimens 20 are similar. Examples of the similar specimens here include a tissue section before staining of a tissue section to be stained (hereinafter, section), a section adjacent to and contiguous with a stained section, a section different from a stained section in the same block (sampled from the same place as that of the stained section), a section in a different block (sampled from a different place from that of a stained section) in the same tissue), and a section collected from a different patient. Therefore, when an autofluorescence spectrum can be extracted from a certain specimen 20, the separation processing unit 132 may extract a fluorescence spectrum from image information of another specimen 20 by removing the autofluorescence spectrum from the image information of the other specimen 20.

In addition, when calculating an S/N value using the image information of the other specimen 20, the separation processing unit 132 can improve the S/N value by using a background after the autofluorescence spectrum is removed.

### Image Generation Unit 133

The image generation unit 133 is configured to generate image information based on the separation result of the linked fluorescence spectrum by the separation processing unit 132. For example, the image generation unit 133 can generate image information using one or more fluorescence spectra corresponding to one or more fluorescent molecules, or generate image information using one or more autofluorescence spectra corresponding to one or more autofluorescent molecules. The number and combination of fluorescent molecules or autofluorescent molecules used by the image generation unit 133 to generate the image information are not particularly limited. In addition, when various types of processing (for example, segmentation, calculation of an S/N value, or the like) using the fluorescence spectrum or the autofluorescence spectrum after the separation are performed, the image generation unit 133 may generate image information indicating the processing results thereof.

### Analysis Unit 134

The analysis unit 134 performs analysis processing according to each embodiment of the present disclosure on the image information generated by the image generation unit 133. For example, the analysis unit 134 performs nucleus detection and cell membrane detection on the image information, and performs cell segmentation. At this time, the analysis unit 134 performs the same analysis processing on the fluorescence-stained specimen 30 and the unstained specimen not stained with the fluorescent sample. The unstained specimen may be one not stained with a substance other than DAPI. The analysis unit 134 calculates an index for performing a positive determination, based on the analysis result for the unstained specimen and the analysis result for the stained specimen. The analysis unit 124 performs a positive determination on the stained specimen, based on the calculated index.

Here, the index calculated by the analysis unit 134 is information that allows a target to be quantitatively evaluated based on a numerical value or the like, and can be used as an objective evaluation value that does not depend on a specific observer for the target (referred to as an objective index). In contrast, an index based on the characteristics of the observer (for example, an index unique to the observer based on the experience of the observer) is referred to as a subjective index, and is distinguished from the objective index.

### Display Section 140

The display section 140 is configured to present the image information generated by the image generation unit 133 to the operator by displaying the image information on a display. The display section 140 may also present, on the display, the analysis and determination result of the analysis unit 134.

Note that the type of display used as the display section 140 is not particularly limited. Although not described in detail in the present embodiment, the image information generated by the image generation unit 133 may be presented to the operator by being projected by a projector or printed by a printer (in other words, a method of outputting the image information is not particularly limited).

### Control Unit 150

The control unit 150 has a functional configuration of comprehensively controlling the overall processing performed by the information processing apparatus 100. For example, the control unit 150 controls the start, end, and the like of various types of processing (for example, processing of adjusting the placement position of the fluorescence-stained specimen 30, processing of irradiating the fluorescence-stained specimen 30 with excitation light, processing of acquiring the spectra, processing of generating the linked fluorescence spectrum, fluorescence separation processing, processing of generating the image information, processing of displaying the image information, and the like) as described above, based on an operation input performed by the operator via the operation section 160. The control contents of the control unit 150 are not particularly limited. For example, the control unit 150 may control processing (for example, processing related to an operating system (OS)) generally performed in a general-purpose computer, a PC, a tablet PC, or the like.

### Operation Section 160

The operation section 160 is configured to receive an operation input from the operator. More specifically, the operation section 160 includes various types of input means such as a keyboard, a mouse, a button, a touch panel, or a microphone, and the operator can perform various inputs to the information processing apparatus 100 by operating the input means. Information related to the operation input performed via the operation section 160 is provided to the control unit 150.

### Database 200

The database 200 is an apparatus that manages the reagent information, the specimen information, and the like. More specifically, the database 200 manages the reagent identification information 11 and the reagent information in association with each other, and the specimen identification information 21 and the specimen information in association with each other. Accordingly, the information acquisition unit 111 can acquire, from the database 200, the reagent information based on the reagent identification information 11 of the fluorescent reagent 10 and the specimen information based on the specimen identification information 21 of the specimen 20.

The reagent information managed by the database 200 is assumed to be information including measurement channels specific to the fluorescent substances of the fluorescent reagent 10 and the linked fluorescence reference spectrum (not necessarily limited thereto). The “measurement channels” are concepts indicating the fluorescent substances contained in the fluorescent reagent 10 and are concepts indicating CK, ER, PgR, and DAPI in the example of FIG. 9. Since the number of fluorescent substances varies depending on the fluorescent reagent 10, the measurement channels are managed as the reagent information in association with each fluorescent reagent 10. The linked fluorescence reference spectrum included in the reagent information is obtained by linking, in the wavelength direction, the fluorescence spectra for each of the fluorescent substances included in the measurement channels as described above.

The specimen information managed by the database 200 is assumed to be information including measurement channels specific to the autofluorescent substances of the specimen 20 and the linked autofluorescence reference spectrum (not necessarily limited thereto). The “measurement channels” are concepts indicating the autofluorescent substances contained in the specimen 20, and are concepts indicating Hemoglobin, Archidonic Acid, Catalase, Collagen, FAD, NADPH, and ProLong Diamond in the example of FIG. 8.

Since the number of autofluorescent substances varies depending on the specimen 20, the measurement channels are managed as the specimen information in association with each specimen 20. As described above, the linked autofluorescence reference spectrum included in the specimen information is obtained by linking, in the wavelength direction, the autofluorescence spectra for each of the autofluorescent substances included in the measurement channels. The information managed by the database 200 is not necessarily limited to the above.

The configuration example of the information processing system applicable to each embodiment of the present disclosure has been described above. Note that the configuration described above with reference to FIG. 1 is a mere example, and the configuration of the information processing system according to the present embodiment is not limited to such an example. For example, the information processing apparatus 100 does not necessarily need to include all the components depicted in FIG. 1, and may include components not depicted in FIG. 1.

Here, the information processing system applicable to each embodiment of the present disclosure may include an imaging apparatus (including, for example, a scanner or the like) that acquires a fluorescence spectrum, and an information processing apparatus that performs processing using the fluorescence spectrum. In this case, the fluorescence signal acquisition unit 112 depicted in FIG. 1 can be implemented by the imaging apparatus, and the other components can be implemented by the information processing apparatus.

In addition, the information processing system applicable to each embodiment of the present disclosure may include an imaging apparatus that acquires a fluorescence spectrum and software used for processing of using the fluorescence spectrum. In other words, the information processing system does not need to include a physical component (for example, a memory, a processor, or the like) that stores or executes the software. In this case, the fluorescence signal acquisition unit 112 depicted in FIG. 1 can be implemented by the imaging apparatus, and the other components can be implemented by the information processing apparatus on which the software is executed.

The software is provided to the information processing apparatus via a network (for example, from a website, a cloud server, or the like) or via any storage medium (for example, a disk or the like).

The information processing apparatus on which the software is executed can be various types of servers (for example, a cloud server or the like), a general-purpose computer, a PC, a tablet PC, or the like. Note that a method of providing the software to the information processing apparatus and the type of information processing apparatus are not limited to the above. In addition, it should be noted that the configuration of the information processing system according to the present embodiment is not necessarily limited to the above, and a configuration that can be conceived of by a person skilled in the art based on the technical level at the time of use can be applied.

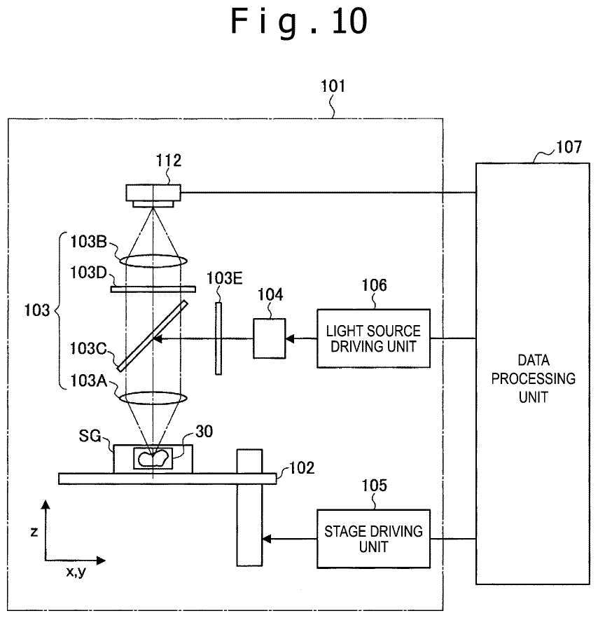

The information processing system described above may be implemented as, for example, a microscope system. Accordingly, a configuration example of the microscope system when the information processing system applicable to each embodiment is implemented as the microscope system will be described with reference to FIG. 10.

As depicted in FIG. 10, the microscope system applicable to each embodiment includes a microscope 101 and a data processing unit 107.

The microscope 101 includes a stage 102, an optical system 103, a light source 104, a stage driving unit 105, a light source driving unit 106, and a fluorescence signal acquisition unit 112.

The stage 102 has a mounting surface on which the fluorescence-stained specimen 30 can be mounted, and is movable in a parallel direction (x-y plane direction) and a perpendicular direction (z-axis direction) with respect to the mounting surface by driving the stage driving unit 105. The fluorescence-stained specimen 30 has a thickness of, for example, several μm to several tens of μm in the Z direction, and is fixed by a predetermined fixing method while being sandwiched between a slide glass SG and a cover glass (not illustrated).

The optical system 103 is disposed above the stage 102. The optical system 103 includes an objective lens 103A, an image formation lens 103B, a dichroic mirror 103C, an emission filter 103D, and an excitation filter 103E. The light source 104 is, for example, an electric bulb such as a mercury lamp, a light emitting diode (LED), or the like, and irradiates a fluorescent label attached to the fluorescence-stained specimen 30 with excitation light by driving the light source driving unit 106.

When a fluorescence image of the fluorescence-stained specimen 30 is obtained, the excitation filter 103E generates excitation light by causing, among light beams emitted from the light source 104, only a light beam having an excitation wavelength for exciting a fluorescent pigment to pass therethrough. The dichroic mirror 103C reflects the excitation light that has passed through the excitation filter and that is incident thereon, and guides the excitation light to the objective lens 103A. The objective lens 103A condenses the excitation light on the fluorescence-stained specimen 30. The objective lens 103A and the image formation lens 103B magnify an image of the fluorescence-stained specimen 30 at a predetermined magnification, and form the magnified image on an imaging surface of the fluorescence signal acquisition unit 112.

When the fluorescence-stained specimen 30 is irradiated with the excitation light, a staining agent bound to each tissue of the fluorescence-stained specimen 30 emits fluorescence. The fluorescence passes through the dichroic mirror 103C via the objective lens 103A, and reaches the image formation lens 103B via the emission filter 103D. The emission filter 103D absorbs the light that has been magnified by the objective lens 103A and that has passed through the excitation filter 103E, and causes only part of the emitted light to pass therethrough. The image of the emitted light from which the external light has been lost is magnified by the image formation lens 103B and formed on the fluorescence signal acquisition unit 112 as described above.

The data processing unit 107 is configured to drive the light source 104, acquire the fluorescence image of the fluorescence-stained specimen 30 using the fluorescence signal acquisition unit 112, and perform various types of processing using the fluorescence image. More specifically, the data processing unit 107 can function as some or all components of the information acquisition unit 111, the storage unit 120, the processing unit 130, the display section 140, the control unit 150, the operation section 160, and the database 200 of the information processing apparatus 100 described with reference to FIG. 1. For example, the data processing unit 107 functions as the control unit 150 of the information processing apparatus 100 to control the driving of the stage driving unit 105 and the light source driving unit 106 and to control the acquisition of a spectrum by the fluorescence signal acquisition unit 112. The data processing unit 107 functions as the processing unit 130 of the information processing apparatus 100 to generate a linked fluorescence spectrum, separate the linked fluorescence spectrum for each molecule, and generate image information based on a result of the separation.

The configuration example of the microscope system when the information processing system applicable to each embodiment of the present disclosure is implemented as the microscope system has been described above. Note that the configuration described above with reference to FIG. 10 is a mere example, and the configuration of the microscope system applicable to each embodiment of the present disclosure is not limited to such an example. For example, the microscope system does not necessarily need to include all of the components depicted in FIG. 10, and may include components not depicted in FIG. 10.

### Processing Flow Example

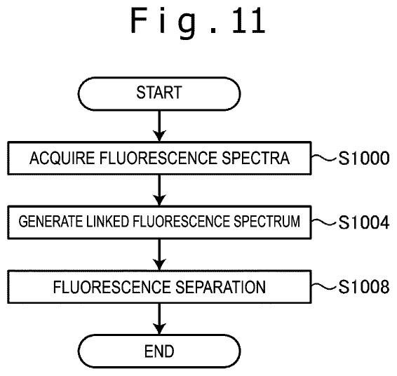

The configuration example of the information processing system applicable to each embodiment of the present disclosure has been described above. Next, an example of a series of processing flow involved in fluorescence separation by the information processing apparatus 100 will be described with reference to FIG. 11. FIG. 11 is an example of a flowchart depicting the example of the series of processing flow involved in the fluorescence separation by the information processing apparatus 100.

In step S1000, the fluorescence signal acquisition unit 112 of the information processing apparatus 100 acquires fluorescence spectra. More specifically, the fluorescence-stained specimen 30 is irradiated with a plurality of excitation light beams having different excitation wavelengths, and the fluorescence signal acquisition unit 112 acquires a plurality of fluorescence spectra corresponding to the respective excitation light beams. Then, the fluorescence signal acquisition unit 112 stores the acquired fluorescence spectra in the fluorescence signal storage unit 122.

In step S1004, the link unit 131 links at least some of the plurality of fluorescence spectra stored in the fluorescence signal storage unit 122 in the wavelength direction to generate a linked fluorescence spectrum. More specifically, the link unit 131 extracts data having a predetermined width from each of the plurality of fluorescence spectra so as to include the maximum value of the fluorescence intensity of each fluorescence spectrum, and links these pieces of data to each other in the wavelength direction to generate one linked fluorescence spectrum.

In step S1008, the separation processing unit 132 separates the linked fluorescence spectrum for each molecule (performs fluorescence separation). More specifically, the separation processing unit 132 separates the linked fluorescence spectrum for each molecule by executing the processing described with reference to FIG. 7.

In the subsequent processing, for example, the image generation unit 133 generates image information using one or more fluorescence spectra corresponding to one or more fluorescent molecules (or autofluorescence spectrum corresponding to an autofluorescent molecule) after the separation, and the display section 140 displays the image information on the display to present the image information to the operator. The analysis unit 134 may perform analysis processing including cell segmentation on the image information generated by the image generation unit 133. The analysis unit 134 may perform a positive determination of a cell based on the result of the analysis processing.

### Hardware Configuration

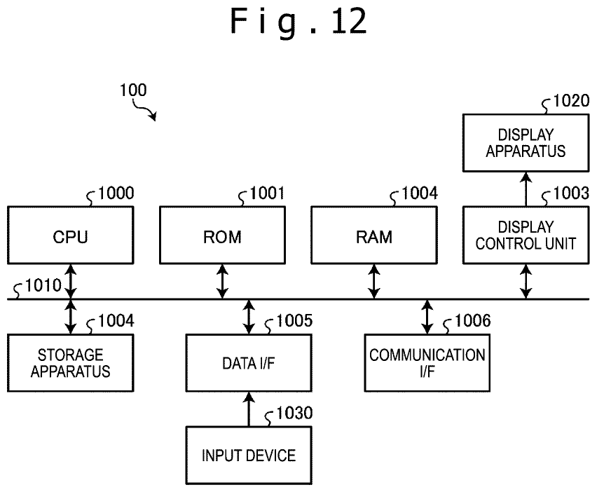

FIG. 12 is a block diagram depicting an example of a hardware configuration of the information processing apparatus 100 applicable to each embodiment.

In FIG. 12, the information processing apparatus 100 includes a CPU 1000, a read only memory (ROM) 1001, a random access memory (RAM) 1002, a display control unit 1003, a storage apparatus 1004, a data I/F 1005, and a communication I/F 1006. These components are communicably connected to each other via a bus 1010.

The storage apparatus 1004 is a nonvolatile storage medium such as a hard disk drive or a flash memory. The CPU 1000 operates, using the RAM 1002 as a work memory, according to programs stored in the ROM 1001 and the storage apparatus 1004, and controls the operation of the information processing apparatus 100.

The display control unit 1003 generates a display signal that can be handled by a display apparatus 1020, based on display control information generated by the CPU 1000 according to a program. The display control unit 1003 outputs the generated display signal to the display apparatus 1020. The display apparatus 1020 includes a display device such as a liquid crystal display (LCD) or an organic electro-luminescence (EL) display, and a drive circuit that drives the display device. The display apparatus 1020 displays a screen on the display device in accordance with the display signal supplied from the display control unit 1003. The display control unit 1003 and the display apparatus 1020 may correspond to the display section 140 described above.

The data I/F 1005 is an interface for transmitting and receiving data to and from an external device. An interface applicable as the data I/F 1005 is not particularly limited, but an interface via wired communication such as a universal serial bus (USB) or wireless communication such as Bluetooth (trade name) may be used. In the example of FIG. 12, an input device 1030 such as a keyboard or a touch panel for a user to input an operation is connected to the data I/F 1005. The input device 1030 may correspond to the operation section 160 described above.

The communication I/F 1006 is an interface for communicating with a communication network such as the Internet or a local area network (LAN).

In the information processing apparatus 100, the CPU 1000 executes an information processing program according to each embodiment, thereby configuring the above-described acquisition unit 110, storage unit 120, processing unit 130, and control unit 150 as, for example, modules in a main storage area of a RAM 2002.

The information processing program can be acquired from the outside via a communication network (not illustrated) through communication via, for example, the communication I/F 1006, and installed on the information processing apparatus 100. The present disclosure is not limited thereto, and the program may be provided by being stored in a removable storage medium such as a compact disk (CD), a digital versatile disk (DVD), or a universal serial bus (USB) memory.

### 3. FIRST EMBODIMENT

Next, a first embodiment of the present disclosure will be described.

In the first embodiment of the present disclosure, two images, that is, an image obtained by imaging a slide stained with only DAPI as an unstained image and an image obtained by multi-staining a target tissue are treated as a set in the same manner, and color separation, autofluorescence difference mask processing, nucleus detection, membrane detection, and luminance (antibody value) calculation are performed.

### 3-1. Processing According to First Embodiment

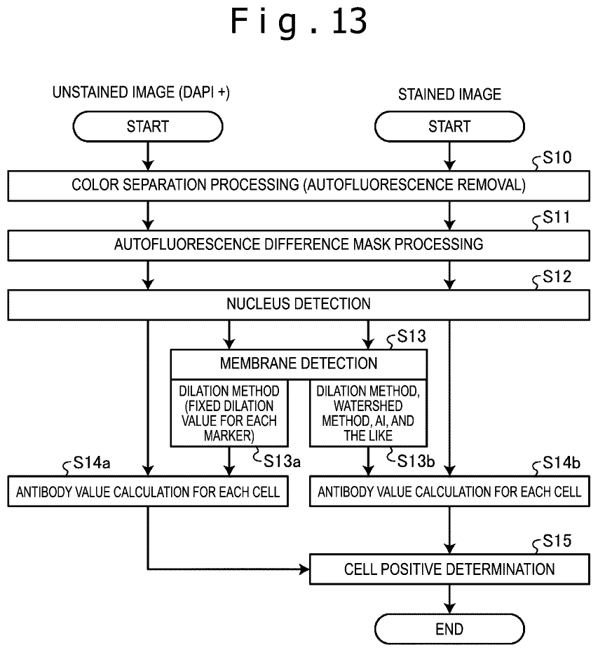

FIG. 13 is an example of a flowchart depicting a positive determination method according to the first embodiment. In the first embodiment, as described above, the same processing is performed on an unstained image obtained by imaging an unstained specimen and a stained image obtained by imaging a fluorescence-stained specimen.

First, the processing on the unstained image will be described. In FIG. 13, the unstained image is obtained by imaging a specimen that contains a target cell and that is stained with only DAPI for nucleus detection. For example, in the configuration of FIG. 1, in step S10, the processing unit 130 causes the separation processing unit 132 to perform color separation processing on the unstained image, and removes autofluorescent components included in the unstained image. In next step S11, the processing unit 130 causes, for example, the image generation unit 133 to perform autofluorescence difference mask processing on the unstained image, based on a result of the color separation in step S10.

In next step S12, the processing unit 130 causes the analysis unit 134 to perform cell segmentation to perform nucleus detection based on a portion stained with DAPI in the unstained image. The analysis unit 134 may perform the nucleus detection by, for example, artificial intelligence (AI) processing using a learning model generated by machine learning (AI nucleus detection).

In next step S13, the analysis unit 134 performs membrane detection on the unstained image. The analysis unit 134 performs the membrane detection on the unstained image by, for example, a dilation method (step S13a). At this time, the analysis unit 134 may perform the membrane detection using a fixed dilation value for each marker in the dilation method.

In next step S14a, the analysis unit 134 calculates an antibody value for each cell, based on the information on the cell nucleus detected in step S12 and the information on the cell membrane detected in step S13a. The antibody value is calculated as, for example, luminance information.

Next, processing on the stained image will be described. According to the present disclosure, the same processing as the processing on the unstained image described above is also performed on the stained image.

In FIG. 13, the stained image is obtained by imaging a specimen that contains the target cell and that is stained with, for example, a plurality of fluorescent reagents. For example, in the configuration of FIG. 1, in step S10, the separation processing unit 132 performs color separation processing on the stained image to remove autofluorescent components included in the stained image. In next step S11, for example, the image generation unit 133 performs autofluorescence difference mask processing on the stained image, based on the result of the color separation in step S10.

Note that the above-described unstained specimen may be a specimen that contains the target cell and that is the same as and/or similar to the specimen.

In next step S12, the analysis unit 134 performs cell segmentation to perform nucleus detection on the stained image. The cell segmentation refers to a process of individually identifying and segmenting cells and intracellular structures in a biological sample. This process aims to detect and extract a cell from a data source such as a microscope image or biometric data, and to separate the cell from the other cells and the background, based on a feature such as the shape, size, luminance, or texture. Cell segmentation technologies range from conventional image processing methods such as threshold processing, edge detection, morphological operation, and the watershed method to methods that utilize deep learning or machine learning in recent years.

In next step S13, the analysis unit 134 performs membrane detection on the stained image. The analysis unit 134 performs the membrane detection on the stained image by, for example, the dilation method (step S13b). The membrane detection is not limited thereto, and the analysis unit 134 may perform the membrane detection by the watershed method or AI processing using a learning model generated by machine learning.

The dilation method is a type of morphological image processing, and refers to an operation of enlarging the boundary of an object on a binary image. The dilation method is used for clarifying the boundary of a cell in cell segmentation. In the dilation method, a predetermined structuring element (kernel) is applied to an object region on an image, and when the object region is within the structuring element, the pixel is processed as belonging to the object region. This operation enlarges the object region and clarifies the boundary between adjacent cells.

The watershed method is an image processing method of dividing an object region with reference to a local minimum value (watershed) of a grayscale image. In this method, the minimum value of the image is regarded as a valley, and the object region is divided by conceptually raising the water level. In the cell segmentation, the watershed method can be applied to find the boundary of a cell. Specifically, a clear portion of the cell is used as a water source, and a region is enlarged in such a manner that water spreads therearound, thereby detecting the boundary with an adjacent cell region.

In next step S14b, the analysis unit 134 calculates an antibody value for each cell based on the information on the cell nucleus detected in step S12 and the information on the cell membrane detected in step S13b. The antibody value is calculated as, for example, luminance information.

In next step S15, the processing unit 130 causes the analysis unit 134 to perform a positive determination on the fluorescence-stained specimen, based on the antibody value calculated from the unstained image in step S14a and the antibody value calculated from the stained image in step S14b. The analysis unit 134 may obtain, for example, a difference between the antibody value calculated from the unstained image and the antibody value calculated from the stained image and determine, based on this difference, a threshold for the positive determination for the stained image.

3-2. Threshold Setting Using Unstained Image without Depending on Subjectivity

In the first embodiment, as described above, analyzing the unstained image and the stained image under the same conditions allows for comparison between the unstained image and the stained image. Therefore, by determining the threshold using the unstained image as a control, the determination of the staining characteristics (positive/negative) of the stained image, which necessarily relies on visual check when only the stained image is used, can be performed based on an objective index.

Here, the color separation processing in step S10 is performed using the least squares method (LSM) or the weighted least squares method (WLSM) as described with reference to FIG. 7. The color separation processing is not limited thereto, and a method using a virtual filter is also known.

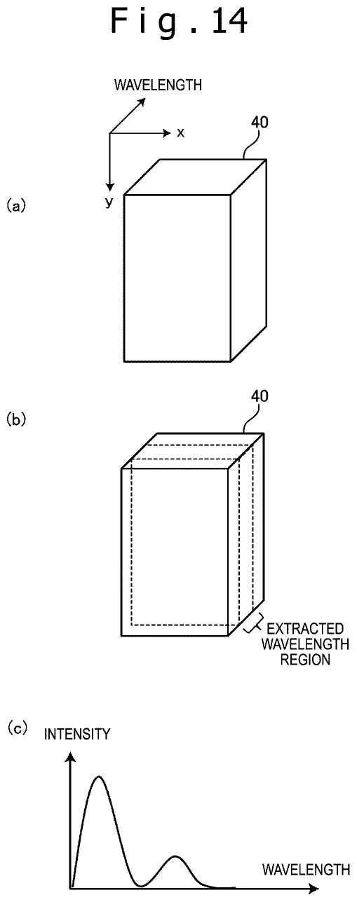

FIG. 14 is a schematic diagram for describing the color separation processing applied to the first embodiment and the color separation processing using the virtual filter. As depicted in Section (a) of FIG. 14, a reference spectrum input to the separation processing unit 132 has a structure called a spectrum cube 40 represented by position information indicated by x-y coordinates and wavelength information in a direction perpendicular to the xy plane.

As depicted in Section (b) of FIG. 14 as an extracted wavelength region, the virtual filter extracts information on a specific wavelength region of the spectrum cube 40, and integrates the extracted information in the wavelength direction, thereby obtaining a virtual filter image. On the other hand, in the color separation processing applied to the first embodiment, as described in JP 2020-144109 A, matrix decomposition is performed using the least squares method to obtain an intensity distribution in the wavelength direction (see Section (c) of FIG. 14), which is then multiplied by a coefficient to thereby obtain a color separation image.

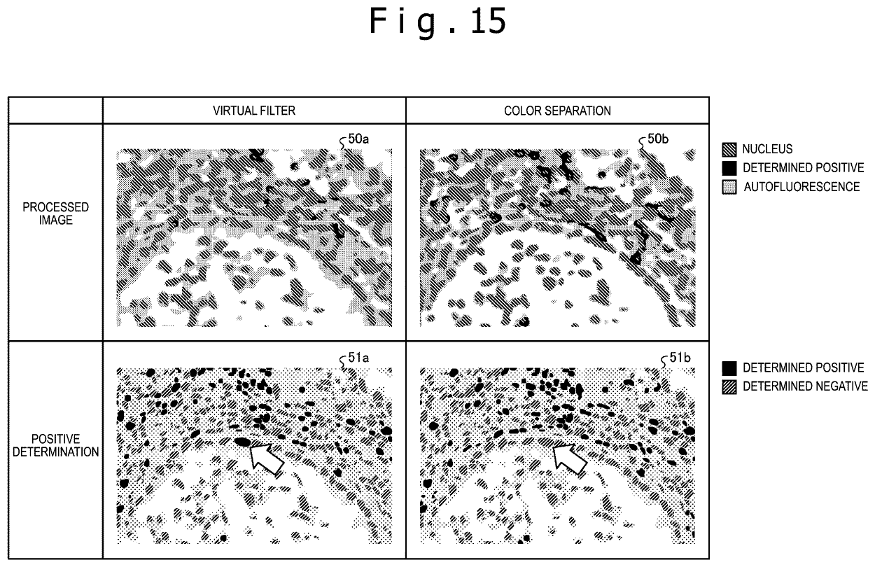

FIG. 15 is a schematic diagram depicting results of performing the color separation using the virtual filter and results of performing by using the color separation applied to the first embodiment in comparison with each other. In FIG. 15, examples (images 50a and 51a) in the case of using the virtual filter are depicted on the left side, and examples (images 50b and 51b) in the case of performing the color separation applied to the first embodiment are depicted on the right side. In addition, in FIG. 15, examples of processed images that have been color-separated are depicted in the upper row, and examples of results upon having performed a positive determination based on a comparison result between a stained image and an unstained image are depicted in the lower row. Note that, in the lower row, a threshold for the positive determination is set so that the numbers of positive cells are equivalent for comparison.

In the upper row of FIG. 15, autofluorescence is not completely removed in the case of performing the color separation using the virtual filter, which is depicted as the image 50a on the left side, and thus it can be confirmed that the background becomes higher than in the case of performing the color separation applied to the first embodiment, which is depicted as the image 50b on the right side.

In addition, in the examples in the lower row, it can be understood that, in the case of performing the color separation using the virtual filter, which is depicted as the image 51a on the left side, the number of false positives tends to increase due to the influence of autofluorescence, compared with the case of performing the color separation applied to the first embodiment, which is depicted as the image 51b on the right side. As an example, referring to portions indicated by arrows in the drawing, the determination is positive for the case on the left side where the virtual filter is used, but the determination is negative for the case on the right side where the color separation applied to the first embodiment is performed.

The occurrence of autofluorescence varies depending on the site, which thus affects and changes the optimal threshold setting. The color separation applied to the first embodiment can simultaneously perform autofluorescence removal, which can reduce such fluctuation.

### 3-3. Positive Determination Method Capable of Suppressing Spatial Leakage of Cell

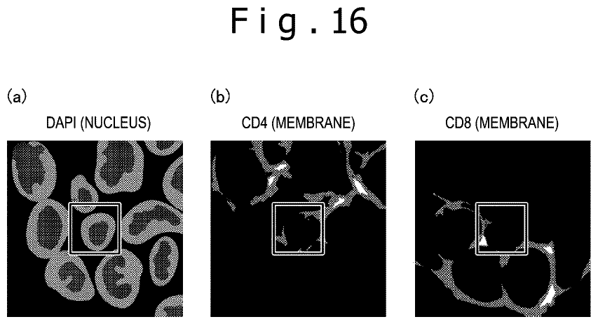

Unlike single-cell analysis using a cell sorter, a cell that should be originally CD4 positive may be erroneously determined to be CD8 positive due to spatial leakage in image analysis of a tissue as depicted in FIG. 16. This is because CD8 positive cells are adjacent to the cell in the vicinity in a state where cells are densely packed.

FIG. 16 is a schematic diagram for describing an erroneous determination due to spatial leakage. In FIG. 16, Section (a) depicts a nucleus detected by DAPI staining. Sections (b) and (c) depict cell membranes detected by membrane detection, Section (b) depicts a site determined to be CD4 positive, and Section (c) depicts a site determined to be CD8 positive.

In the drawing, the cells in frames are considered to be positive in the case of CD4 and negative in the case of CD8. However, in the determination of CD8 depicted in Section (c), cells around a target cell are stained. Thus, when the average of the luminance values in the frame is calculated, the cell in the frame is determined to be positive depending on the setting of the threshold. That is, in the example of Section (c), the target cell is erroneously determined due to spatial leakage of the staining results of the cells adjacent to the target cell.

In the first embodiment, the analysis unit 134 performs a positive determination using any of the following four methods.
(a) Average luminance method
(b) Positive pixel method
(c) Combination of the average luminance method and the positive pixel method
(d) On-membrane luminance continuity calculation method

### (a) Average Luminance Method

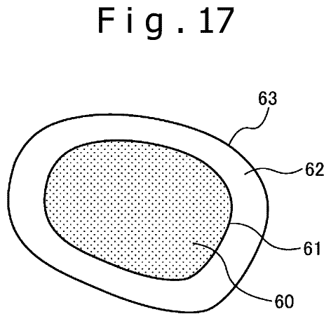

This is a method generally used as a method of determining a positive cell in the existing technology. FIG. 17 is a schematic diagram for describing the average luminance method according to the existing technology. In FIG. 17, a nucleus contour 61 is detected by AI nucleus detection based on, for example, DAPI staining (step S12 of FIG. 13), and a membrane contour 63 is calculated from the nucleus contour 61 by, for example, the dilation method (step S13 of FIG. 13). The inside of the nucleus contour 61 is a cell nucleus 60. The analysis unit 134 extracts a region 62 between the nucleus contour 61 and the membrane contour 63, and performs a positive determination, using a threshold, on the average luminance obtained by averaging the luminance of the region 62.

### (b) Positive Pixel Method

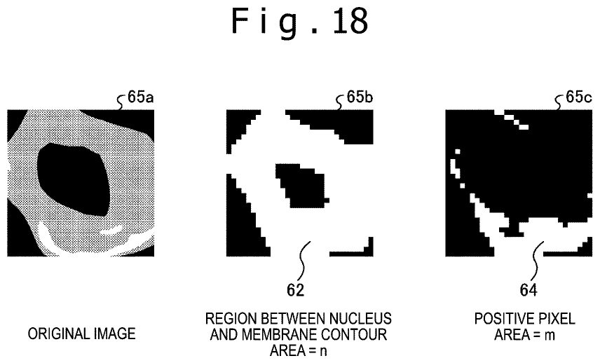

The positive pixel method is a method newly proposed as a method of determining a positive cell in the first embodiment. FIG. 18 is a schematic diagram for describing the positive pixel method according to the first embodiment.

In the positive pixel method, the analysis unit 134 first determines a positive pixel on a pixel-by-pixel basis in an original image 65a, which is a stained image, using a threshold determined from a negative control or the like using an unstained image, for example. Next, the analysis unit 134 obtains the number n of pixels in the region 62 between the nucleus contour 61 and the membrane contour 63 as depicted in an image 65b by nucleus detection and membrane detection. Further, as depicted in an image 65c, the analysis unit 134 obtains the number m of positive pixels included in a positive pixel region 64 in the region 62. The analysis unit 134 calculates a ratio (m/n) of the positive pixel region 64 in the region 62, and determines that the cell included in the positive pixel region 64 is a positive cell when the calculated ratio (m/n) is equal to or greater than a threshold. That is, in the positive pixel method, the ratio (m/n) may be an objective index for the positive determination.

### (c) Combination of Average Luminance Method and Positive Pixel Method

This is a method combining (a) the average luminance method and (b) the positive pixel method described above. The analysis unit 134 determines that the cell is a positive cell when the average luminance obtained by (a) the average luminance method is equal to or greater than the threshold and the ratio (m/n) obtained by (b) the positive pixel method is equal to or greater than the threshold.

### (d) On-Membrane Luminance Continuity Calculation Method

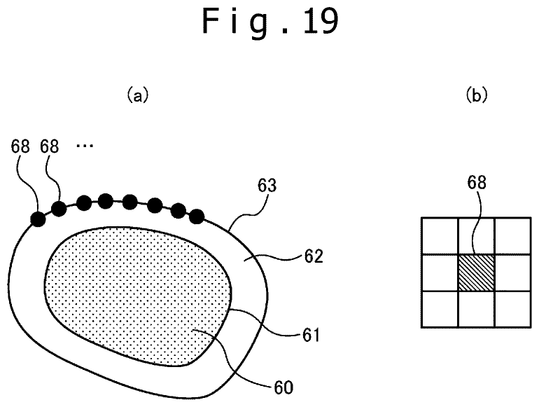

The on-membrane luminance continuity calculation method is a method newly proposed as a method of determining a positive cell in the first embodiment. FIG. 19 is a schematic diagram for describing the on-membrane luminance continuity calculation method according to the first embodiment.

In the on-membrane luminance continuity calculation method, the analysis unit 134 obtains the luminance of each of membrane contour points 68 corresponding to pixels on the membrane contour 63 as depicted in Section (a) of FIG. 19. At this time, as depicted in Section (b) of FIG. 19, the average luminance of pixels including a pixel of each membrane contour point 68 and, for example, nine pixels around the membrane contour point 68 is calculated, and the calculated average luminance value is used as the luminance value of the pixel of the membrane contour point 68.

The analysis unit 134 calculates the luminance value of each membrane contour point 68 over the entire membrane contour 63. The analysis unit 134 determines that the cell related to the membrane contour 63 is a positive cell when the membrane contour 63 is consecutively viewed and a predetermined number or more of luminance values do not consecutively fall below a threshold. That is, when the number of consecutive membrane contour points 68 having a luminance value equal to or less than the threshold does not exceed the predetermined number, the analysis unit 134 determines that the cell related to the membrane contour 63 is a positive cell. In other words, it can be said that, when the number of membrane contour points 68 having a luminance value equal to or less than the threshold exceeds the predetermined number, the cell related to the membrane contour 63 is determined to be not a positive cell.

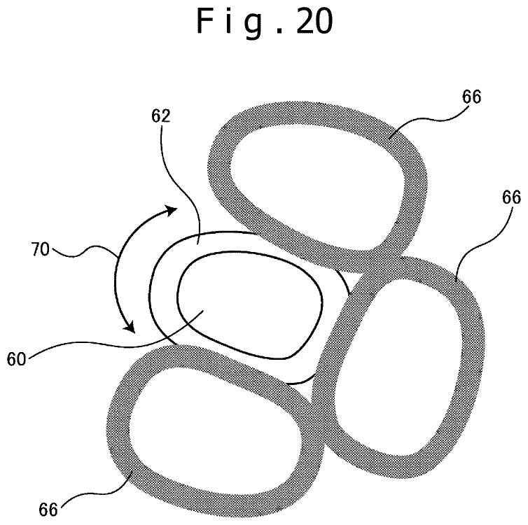

FIG. 20 is a schematic diagram for more specifically describing the on-membrane luminance continuity calculation method according to the first embodiment. In FIG. 20, each of regions 66 is a region between a nucleus contour and a membrane contour of a positively determined cell determined to be positive and emits fluorescence due to staining. In a cell nucleus 60, a portion that is included in a region 62 related to the cell nucleus 60 and that is adjacent to the regions 66 is highly likely to be determined to be positive due to leakage of fluorescence from the regions 66.

On the other hand, a range 70 of the cell nucleus 60 is not adjacent to the regions 66, and thus, fluorescence does not leak or the influence of leakage is extremely small, and the luminance value related to the fluorescence is low. In the on-membrane luminance continuity calculation method, when the ratio of the range 70 in the entire contour of the cell nucleus 60 is equal to or less than a predetermined value, a cell related to the cell nucleus 60 is determined to be a positive cell.

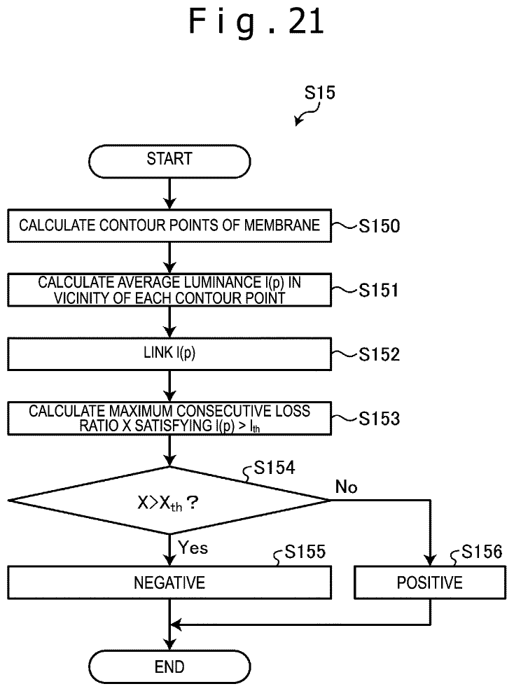

FIG. 21 is an example of a flowchart depicting processing by the on-membrane luminance continuity calculation method according to the first embodiment. The processing according to the flowchart of FIG. 21 corresponds to the processing in step S15 in the flowchart of FIG. 13.

In step S150, the analysis unit 134 calculates contour points of a cell membrane. The analysis unit 134 calculates, for example, the positions of the contour points in the cell membrane detected by the membrane detection in step S13 of FIG. 13. In next step S151, the analysis unit 134 calculates the luminance value l(p) of a pixel at each contour point p. At this time, as described with reference to FIG. 19, the analysis unit 134 calculates, as the luminance value l(p) of the pixel of interest, the average value of the luminance value of the pixel of interest and the luminance values of a plurality of pixels in the vicinity of the pixel of interest.

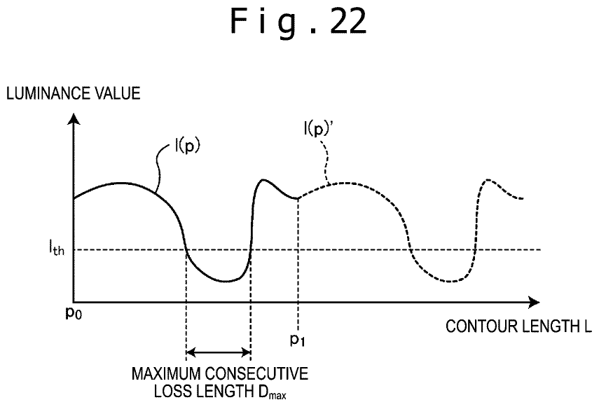

In next step S152, the analysis unit 134 links the luminance values l(p). FIG. 22 is a schematic diagram for describing the linking of the luminance values. In FIG. 22, the horizontal axis represents the length in the contour direction (contour length L) when a given contour point p₀ of the membrane contour is set as a start point, and the vertical axis represents the luminance value l(p).

The analysis unit 134 calculates the luminance value l(p) of each contour point p from, for example, the contour point p₀, which is the start point, to a contour point p₁, which is an end point, at a position reached after one full loop along the membrane contour from the start point. In this case, in order to evaluate one full loop of the contour including the start point and the end point, the luminance value l(p₀) of the start point (contour point p₀) and the luminance value l(p₀) of the end point (contour point p₁) are linked. In the example of FIG. 22, the luminance values l(p) from the contour points p₀ to p₁ are duplicated, for example, and linked as luminance values l(p)′ as indicated by a dotted line. Thus, the luminance values l(p) of the contour points p that are consecutive for one full loop along the contour can be obtained.

In next step S153, the analysis unit 134 obtains a maximum consecutive length L_{con}, which is a length of consecutive contour points p having a luminance value l(p) exceeding a threshold lₜₕ, based on the luminance values l(p) of the respective contour points that are consecutive in one full loop along the contour calculated in step S152. The analysis unit 134 calculates the maximum consecutive loss ratio X by following Equation (10), based on the obtained maximum consecutive length L_{con} and the contour length L_{ALL} of the entire membrane contour.

$$X = {L}_{con} / {L}_{ALL} \qquad ( 10 )$$

The analysis unit 134 determines whether the maximum consecutive loss ratio X calculated in step S153 exceeds a threshold Xₜₕ (X>Xₜₕ?) in next step S154.

Upon determining that the maximum consecutive loss ratio X exceeds the threshold Xₜₕ (“Yes” in step S145), the analysis unit 134 transitions the processing to step S155 and determines that the cell is negative. On the other hand, upon determining that the maximum consecutive loss ratio X is equal to or less than the threshold Xₜₕ (“No” in step S154), the analysis unit 134 transitions the processing to step S156 and determines that the cell is positive. That is, in the on-membrane luminance continuity calculation method, the maximum consecutive loss ratio X may be an objective index for positive determination.

### 3-4. Examples of Determination Results by Each Positive Determination Method

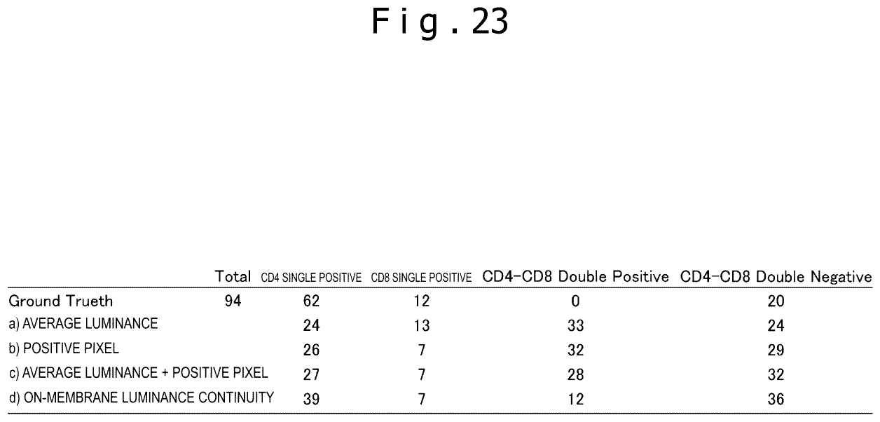

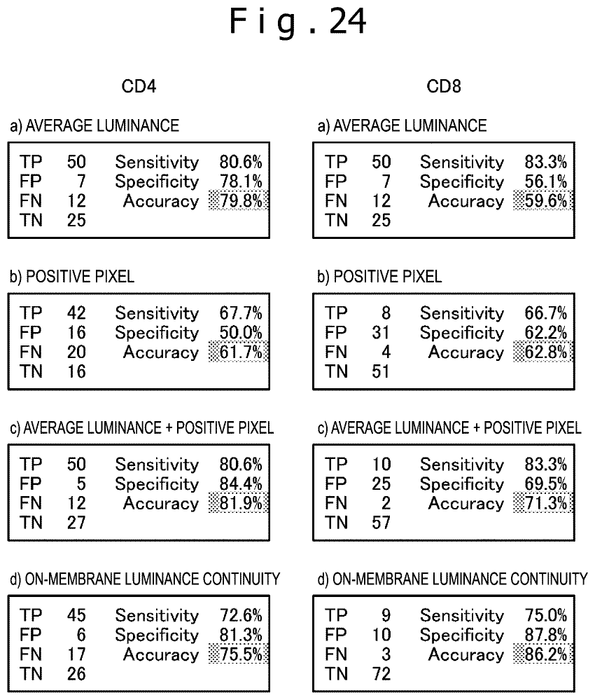

Examples of the determination results obtained by each above-described positive determination method will be described with reference to FIGS. 23 and 24.

FIG. 23 is a schematic diagram depicting actual data examples of the determination results by each positive determination method. FIG. 23 depicts examples of the numbers of detected CD4 single positive cells, CD8 single positive cells, CD4-CD8 double positive cells, and CD4-CD8 double negative cells in each of (a) the average luminance method, (b) the positive pixel method, (c) the combination of the average luminance method and the positive pixel method, and (d) the on-membrane luminance continuity calculation method. In FIG. 23, the unit is “pieces”, and the Ground Truth indicates true values.

In the examples of FIG. 23, the detection result of CD4-CD8 double positive obtained by (d) the on-membrane luminance continuity calculation method has higher accuracy than the others. Accordingly, it can be estimated that (d) the on-membrane luminance continuity calculation method has an effect of suppressing the influence of spatial leakage.

FIG. 24 is a schematic diagram depicting examples of true positive (TP), false positive (FP), false negative (FN), and true positive (TN), as well as sensibility, specificity, and accuracy in each of (a) the average luminance method, (b) the positive pixel method, (c) the combination of the average luminance method and the positive pixel method, and (d) the on-membrane luminance continuity calculation method.

In the examples of FIG. 24, regarding the positive determination of CD4, it is understood that (c) the combination of the average luminance method and the positive pixel method has high values in all of sensibility, specificity, and accuracy, and has the best result among the four types of positive determination methods. On the other hand, regarding the positive determination of CD8, it is similarly understood that (d) the on-membrane luminance continuity calculation method has the best result.

The results depicted in FIG. 24 can be calculated by the analysis unit 134 in any combination based on, for example, the results in step S14a and step S14b in the flowchart of FIG. 13. For example, in the examples of FIG. 24, regarding the positive determination of CD4, (c) the combination of the average luminance method and the positive pixel method has the best result among the four positive determination methods, but the analysis unit 134 may calculate a determination result by combining two or more of (a) the average luminance method, (b) the positive pixel method, and (d) the on-membrane luminance continuity calculation method.

In addition, the information processing apparatus 100 according to the first embodiment can cause the display section 140 to display the determination result of the analysis unit 134 on the display apparatus 1020. The information processing apparatus 100 may present, to the user, a user interface (UI) for causing the user to designate a combination of positive determination methods according to the determination results by the respective positive determination methods displayed on the display apparatus 1020. The analysis unit 134 may calculate the determination result according to the combination of the positive determination methods designated by the user using the UI, and the display section 140 presents the determination result to the user, for example.

As described above, according to the first embodiment, the color separation and analysis processing are performed on the unstained image and the stained image under the same conditions, so that conditions for a negative control can be matched. Therefore, the calculation result of the negative control can be set as a positive determination threshold for the stained image. Since a threshold based on an objective numerical value by the negative control can be adopted, the application of the image processing system according to the first embodiment makes it unnecessary to determine a threshold while subjectively viewing an image, and makes it possible to contribute to reproducibility independent of a doctor (user) and reduction of time taken for the doctor to make a determination.

Further, according to the first embodiment, the calculation is performed for each cell in consideration of leakage from an adjacent cell, and thus it is possible to more accurately determine the positivity of each cell. Therefore, the accuracy of the positive determination can be further enhanced. By applying the image processing system according to the first embodiment, it is possible to perform a positive determination with high accuracy using a tissue as is (using a captured tissue image as is) without sorting cells one by one. This is useful for obtaining a new index such as a spatial positional relationship of a cell population.

In contrast, PTL 1, PTL 2, and PTL 3 describe technologies for normalizing only a stained image without using a control, or for matching conditions even when a control is used. For example, PTL 1 describes a method of determining a threshold by normalizing expression levels in a plurality of regions of interest (ROIs), but is not a method using a control or the like. PTL 2 describes that it is necessary to normalize an expression level, but does not describe an objective index such as use of a control. In addition, PTL 3 describes a method of calculating a threshold from a negative control, but does not describe a mode of performing the same analysis on both a stained image and an unstained image.

In addition, PTL 1 to PTL 3 do not mention spatial leakage between adjacent cells.

In this way, in PTL 1 to PTL 3, the conditions of the negative control are not matched with those of the stained image. Thus, although the threshold can be objectively set to some extent, it is considered to be difficult to apply the same method to different tissues. In the positive determination method according to the first embodiment of the present disclosure, the same color separation processing and analysis processing are applied to the unstained image and the stained image, which allows functioning as a control. In addition, in the positive determination method according to the first embodiment of the present disclosure, spatial leakage between adjacent cells, which is not considered in PTL 1 to PTL 3, is considered. Thus, it is possible to improve the accuracy compared to the positive determination method according to the existing technology.

### 4. SECOND EMBODIMENT

Next, a second embodiment of the present disclosure will be described.

It is conceivable that, among positive determinations with high accuracy, there is a suitable method depending on use, such as whether it is desired to reduce false positives or false negatives. In the second embodiment of the present disclosure, an actual positive determination is performed by combining optimal methods corresponding to use, and thus an effect more suitable for the purpose is obtained.

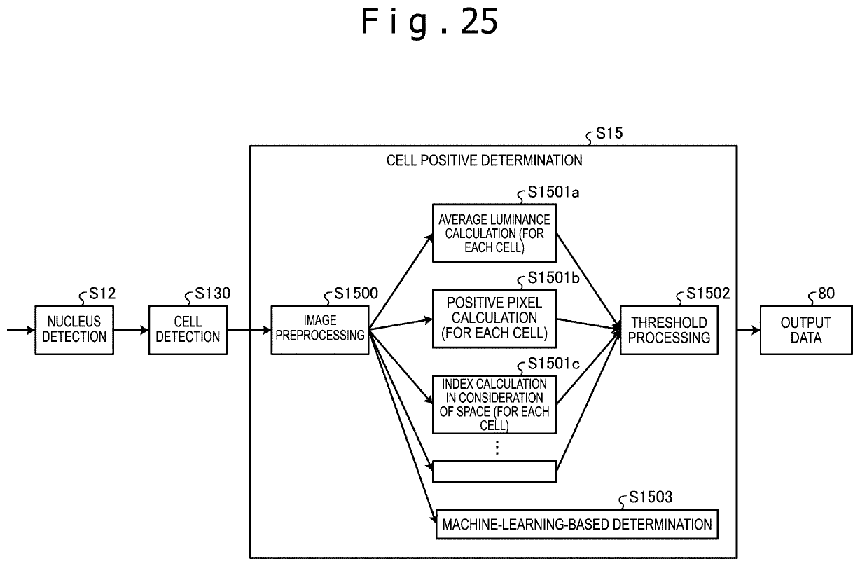

FIG. 25 is a schematic diagram for describing an example of the flow of positive determination processing according to the second embodiment. In FIG. 25, an image processing system according to the second embodiment performs nucleus detection by the processing in step S12 in the flowchart of FIG. 13, and then performs cell detection (step S130). The processing in step S13 may correspond to the processing in step S13 in the flowchart of FIG. 13. After the processing in step S130, the image processing system performs antibody value calculation processing (not illustrated) corresponding to step S14a and step S14b in the flowchart of FIG. 13, and transitions the processing to positive determination processing in step S15.

In step S1500 of the positive determination processing in step S15, the image processing system causes, for example, the analysis unit to execute image preprocessing on a stained image and an unstained image that have been subjected to nucleus detection and cell detection. The image preprocessing may be, for example, processing of dealing with leakage of autofluorescence or the like between adjacent cells, or processing of dealing with non-specific adsorption.

In the image processing system, the analysis unit 134 passes the image preprocessed in step S1500 to any one or two or more among the respective processing of average luminance calculation processing for each cell (step S1501a), positive pixel calculation processing for each cell (step S1501b), index calculation processing in consideration of spatial leakage for each cell (step S1501c), and the like, and machine-learning-based determination processing (step S1503).

Note that the average luminance calculation processing for each cell in step S1501a corresponds to (a) the average luminance method described above. The positive pixel calculation processing for each cell in step S1501b corresponds to (b) the positive pixel method described above. The index calculation processing in consideration of spatial leakage for each cell in step S1501c corresponds to (c) the on-membrane luminance continuity calculation method described above.

The analysis unit 134 performs, in step S1502, threshold processing on the processing results in steps S1501a to 1501c to perform a positive determination. At this time, the analysis unit 134 may select positive determination processing corresponding to the content of the positive determination from among the positive determination processing operations in steps S1501a to 1501c, and perform a threshold determination on the processing result. At this time, the analysis unit 134 may combine the processing results of a plurality of positive determination processing operations among the positive determination processing operations in steps S1501a to 1501c to perform the threshold determination. The analysis unit 134 may select positive determination processing to be used for the threshold determination according to an instruction of the user, or may select based on each processing result and perform the processing again.

On the other hand, in the machine-learning-based determination processing in step S1503, the positive determination is performed by AI processing using a learning model generated by machine learning.

The analysis unit 134 outputs, as output data 80, the positive determination result by the threshold processing in step S1502 or the positive determination result by the machine-learning-based determination processing in step S1503. For example, the display section 140 displays the output data 80 on the display apparatus 1020, and presents the output data 80 to the user.

In the above description, the image preprocessing in step S1500 is performed after the cell detection processing in step S130, but is not limited to this example. For example, the image preprocessing in step S1500 may be performed before the processing in step S12 or between the processing in step S12 and the processing in step S130.

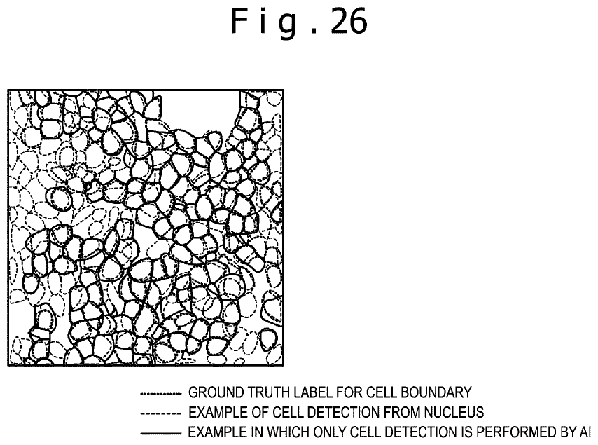

In the example of FIG. 25, nucleus detection is performed in step S12 and then cell detection is performed in step S130, but the cell detection (cell segmentation) can be performed without performing the nucleus detection. FIG. 26 is a schematic diagram depicting an example of a detection result when cell detection is performed without performing nucleus detection, and depicts ground truth labels for cell boundaries, results obtained by performing cell detection from cell nuclei, and results obtained by performing only cell detection by AI (machine-learning-based determination) in comparison with each other.

In the examples of FIG. 26, the example in which only cell detection is performed by AI exhibits high conformity to the ground truth levels. In this way, when a method exhibiting high conformity to the ground truth labels is used, the index calculated by the on-membrane luminance continuity calculation method is considered to be more effective. In this case, it is preferable to select the index calculation in consideration of the space in step S1501c, for example, in the configuration of FIG. 25. The present disclosure is not limited thereto, and it is also conceivable to perform a positive cell determination using AI detection reliability of membrane detection itself.

In this way, in the second embodiment, since a method corresponding to use can be selected from the plurality of positive determination methods, actual positive determination can be executed with higher accuracy.

Note that the effects described in the present specification are mere examples, effects are not limited thereto, and other effects may be added.

Note that the present technology can also have the following configurations.

(1)

A positive determination method including
a first analysis step of performing first analysis processing including cell detection on a stained image obtained by imaging a stained specimen prepared by staining a specimen containing a target cell with a fluorescent reagent,
a second analysis step of performing second analysis processing, which is the same as the first analysis processing, on an unstained image obtained by imaging an unstained specimen, the unstained specimen containing the target cell and being the same as and/or similar to the specimen, the first analysis step and the second analysis step being executed by a processor, and
outputting an index indicating whether or not the target cell is a positive cell, based on a difference between an analysis result of the first analysis processing and an analysis result of the second analysis processing.
   (2)

The positive determination method according to (1) described above, wherein
one of the first analysis processing and the second analysis processing includes at least one of cell segmentation and antibody value calculation.
   (3)

The positive determination method according to (1) or (2) described above, including
calculating the index in consideration of leakage from a positive portion to a negative portion for a respective cell extracted by the cell detection.
   (4)

The positive determination method according to (3) described above, including
using, as the objective index, a ratio of a region of a positive pixel determined for the stained image based on a threshold determined based on the unstained image with respect to a region included in a region between a nucleus contour and a membrane contour based on the cell.
   (5)

The positive determination method according to (4) described above, including
using, as the index, the ratio and an average luminance of the region between the nucleus contour and the membrane contour based on the cell in the stained image.
   (6)

The positive determination method according to (3) described above, wherein
the determination step includes calculating the objective index based on a length of consecutive points having a luminance value equal to or less than a predetermined value in the membrane contour based on the cell.
   (7)

The positive determination method according to (6) described above, including
determining that a cell related to the membrane contour is a positive cell when the length is equal to or less than a threshold.
   (8)

The positive determination method according to (6) or (7) described above, including
obtaining the length by linking an acquisition start point and an acquisition end point of the luminance value in the membrane contour.
   (9)

The positive determination method according to (3) described above, including
using, as the index, an average luminance of the region between the nucleus contour and the membrane contour based on the cell in the stained image.
   (10)

The positive determination method according to any of (1) to (9) described above, wherein
each of the first analysis step and the second analysis step includes
   removing an autofluorescent component of a corresponding one of the stained image and the unstained image by performing color separation using matrix decomposition by a least squares method on the corresponding one of the stained image and the unstained image, and performing a corresponding one of the first analysis processing and the second analysis processing on the corresponding one of the stained image and the unstained image from which the autofluorescent component has been removed.
      (11)

The positive determination method according to any of (1) to (10) described above, including
presenting a list of respective determination results of the positive determinations by a plurality of types of determination methods.
   (12)

The positive determination method according to any of (1) to (11) described above, including
calculating the index based on an analysis result in each of the first analysis step and the second analysis step in which preprocessing is performed, and
selecting a calculation method for calculating the index depending on a method of the cell detection used in the first analysis step and the second analysis step.
   (13)

The positive determination method according to (12) described above, including
performing the cell detection using a learning model generated by machine learning in the first analysis step and the second analysis step, and
performing the positive determination based on reliability of the cell detection in at least one of the first analysis step and the second analysis step.
   (14)

The positive determination method according to (12) described above, including
further performing threshold processing on the index.
   (15)

The positive determination method according to any of (12) to (14) described above, wherein
the first analysis step and the second analysis step include performing the cell detection without performing nucleus detection.
   (16)

An image analysis system including
an imaging apparatus configured to image a specimen and output an image,
an information processing apparatus including
   an analysis unit configured to perform analysis processing including cell detection on the image, and
   a determination unit configured to perform a positive determination based on a result of the analysis processing, and
a presentation unit configured to present a result of the positive determination, wherein
the determination unit is configured to output an index indicating whether or not a target cell is a positive cell, based on a difference between a first analysis result and a second analysis result, the first analysis result being obtained by performing the analysis processing on a stained image obtained by imaging a stained specimen prepared by staining a specimen containing the target cell with a fluorescent reagent, the second analysis result being obtained by performing the same analysis processing as the analysis processing on an unstained image obtained by imaging an unstained specimen, the unstained specimen containing the target cell and being the same as and/or similar to the specimen.
   (17)

An information processing apparatus including
an analysis unit configured to perform analysis processing including cell detection on an image obtained by imaging a specimen, and
a determination unit configured to perform a positive determination based on a result of the analysis processing, wherein
the determination unit is configured to output an index indicating whether or not a target cell is a positive cell, based on a difference between a first analysis result and a second analysis result, the first analysis result being obtained by performing the analysis processing on a stained image obtained by imaging a stained specimen prepared by staining a specimen containing the target cell with a fluorescent reagent, the second analysis result being obtained by performing the same analysis processing as the analysis processing on an unstained image obtained by imaging an unstained specimen, the unstained specimen containing the target cell and being the same as and/or similar to the specimen.

### REFERENCE SIGNS LIST

40 Spectrum cube
50a, 50b, 51a, 51b, 65b, 65c Image
60 Cell nucleus
61 Nucleus contour
63 Membrane contour
68 Membrane contour point
100 Information processing apparatus
130 Processing unit
132 Separation processing unit
133 Image generation unit
134 Analysis unit
140 Display section

1. A positive determination method, comprising:
a first analysis step of performing first analysis processing including cell detection on a stained image obtained by imaging a stained specimen prepared by staining a specimen containing a target cell with a fluorescent reagent;
a second analysis step of performing second analysis processing, which is the same as the first analysis processing, on an unstained image obtained by imaging an unstained specimen, the unstained specimen containing the target cell and being the same as and/or similar to the specimen, the first analysis step and the second analysis step being executed by a processor; and
outputting an index indicating whether or not the target cell is a positive cell, based on a difference between an analysis result of the first analysis processing and an analysis result of the second analysis processing.

2. The positive determination method according to claim 1, wherein
one of the first analysis processing and the second analysis processing includes at least one of cell segmentation and antibody value calculation.

3. The positive determination method according to claim 1, comprising:
calculating the index in consideration of leakage from a positive portion to a negative portion for a respective cell extracted by the cell detection.

4. The positive determination method according to claim 3, comprising:
using, as the index, a ratio of a region of a positive pixel determined for the stained image based on a threshold determined based on the unstained image with respect to a region included in a region between a nucleus contour and a membrane contour based on the cell.

5. The positive determination method according to claim 4, comprising:
using, as the index, the ratio and an average luminance of the region between the nucleus contour and the membrane contour based on the cell in the stained image.

6. The positive determination method according to claim 3, comprising:
calculating the index based on a length of consecutive points having a luminance value equal to or less than a predetermined value in the membrane contour based on the cell.

7. The positive determination method according to claim 6, comprising:
determining that a cell related to the membrane contour is a positive cell when the length is equal to or less than a threshold.

8. The positive determination method according to claim 6, comprising:
obtaining the length by linking an acquisition start point and an acquisition end point of the luminance value in the membrane contour.

9. The positive determination method according to claim 3, comprising:
using, as the index, an average luminance of the region between the nucleus contour and the membrane contour based on the cell in the stained image.

10. The positive determination method according to claim 1, wherein
each of the first analysis step and the second analysis step includes
removing an autofluorescent component of a corresponding one of the stained image and the unstained image by performing color separation using matrix decomposition by a least squares method on the corresponding one of the stained image and the unstained image, and performing a corresponding one of the first analysis processing and the second analysis processing on the corresponding one of the stained image and the unstained image from which the autofluorescent component has been removed.

11. The positive determination method according to claim 1, comprising:
presenting a list of respective determination results of positive determinations by a plurality of types of determination methods.

12. The positive determination method according to claim 1, comprising:
calculating the index based on an analysis result of each of the first analysis step and the second analysis step in which preprocessing is performed; and
selecting a calculation method for calculating the index depending on a method of the cell detection used in the first analysis step and the second analysis step.

13. The positive determination method according to claim 12, comprising:
performing the cell detection using a learning model generated by machine learning in the first analysis step and the second analysis step; and
using, as the index, reliability of the cell detection in at least one of the first analysis step or the second analysis step.

14. The positive determination method according to claim 12, comprising:
further performing threshold processing on the index.

15. The positive determination method according to claim 12, wherein
the first analysis step and the second analysis step include performing the cell detection without performing nucleus detection.

16. An image analysis system comprising:
an imaging apparatus configured to image a specimen and output an image; and
an information processing apparatus including
an analysis unit configured to perform analysis processing including cell detection on the image, and
a determination unit configured to perform a positive determination based on a result of the analysis processing, wherein
the determination unit is configured to output an index indicating whether or not a target cell is a positive cell, based on a difference between a first analysis result and a second analysis result, the first analysis result being obtained by performing the analysis processing on a stained image obtained by imaging a stained specimen prepared by staining a specimen containing the target cell with a fluorescent reagent, the second analysis result being obtained by performing the same analysis processing as the analysis processing on an unstained image obtained by imaging an unstained specimen, the unstained specimen containing the target cell and being the same as and/or similar to the specimen.

17. An information processing apparatus, comprising:
an analysis unit configured to perform analysis processing including cell detection on an image obtained by imaging a specimen; and
a determination unit configured to perform a positive determination based on a result of the analysis processing, wherein
the determination unit is configured to output an index indicating whether or not a target cell is a positive cell, based on a difference between a first analysis result and a second analysis result, the first analysis result being obtained by performing the analysis processing on a stained image obtained by imaging a stained specimen prepared by staining a specimen containing the target cell with a fluorescent reagent, the second analysis result being obtained by performing the same analysis processing as the analysis processing on an unstained image obtained by imaging an unstained specimen, the unstained specimen containing the target cell and being the same as and/or similar to the specimen.
